(19) United States
(12) Patent Application Publication
(10) Pub. No.: US 2025/0051269 A1
(43) Pub. Date: Feb. 13, 2025

(54) PROCESS OF THE PREPARATION OF HYDROXYLAMINE DERIVATIVES

(71) Applicant: Intervet Inc., Rahway, NJ (US)

(72) Inventors: Christophe Pierre Alain Chassaing, Ingelheim am Rhein (DE); Thomas Dahmen, Partenheim (DE); Claudia Scheipers, Ingelheim (DE)

(73) Assignee: Intervet Inc., Rahway, NJ (US)

(21) Appl. No.: 18/721,905

(22) PCT Filed: Dec. 23, 2023
(86) PCT No.: PCT/EP2022/087702

§ 371 Date: Jun. 20, 2024

(30) Foreign Application Priority Data
Dec. 24, 2021 (EP) 21217708.3

(51) Int. Cl. C07C 269/06; C07C 211/27; C07C 211/40; C07C 211/45; C07C 259/06; C07C 269/04; C07C 271/20; C07D 213/38

(52) U.S. Cl. CPC C07C 269/06; C07C 211/27; C07C 211/40; C07C 211/45; C07C 259/06; C07C 269/04; C07C 271/20; C07D 213/38; C07C 2601/02

(57) ABSTRACT

A method of preparing a compound of Formula (I) or salt thereof, with reduced carboxylic acid by-product production.

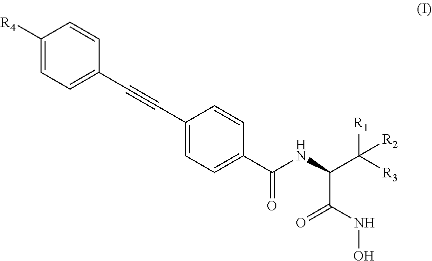

### FIELD OF THE INVENTION

Improved process to prepare compounds which are hydroxylamine derivatives.

### BACKGROUND

Hydroxamic acid antibiotics are small-molecule inhibitors of the enzyme responsible for the biosynthesis of lipid A in Gram-negative organisms (See Kalinin DV, Holl R. Expert Opin Ther Pat. 2017 Nov.; 27(11):1227-1250. doi: 10.1080/13543776.2017.1360282. Epub 2017 Aug. 4. PMID: 28742403).

WO 2018/115432 discloses compounds which are hydroxamic acid antibiotics and that are useful in the treatment of respiratory diseases of animals, especially Bovine or Swine Respiratory disease (BRD and SRD). Examples 1Q, 1R and 1S disclose processes to make these compounds where the hydroxamic acid group is formed at the final or penultimate step of the synthesis.

EP 3 750 881 discloses antibiotic compounds that exhibit excellent antibacterial activity, especially against Gram bacteria and their preparation. Specifically, the conversion of the ester intermediate to the corresponding hydroxamic acid final product is disclosed.

Mohammad A. Alam, “Methods for Hydroxamic Acid Synthesis”, Curr Org Chem. 2019; 23(9): 978-993, discloses methods of making hydroxamic acid compounds from ester and carboxylic acid precursors.

Fei, et al, A, Org. Process Res. Dev. 2012, 16, 1436-1441 discloses a synthetic route to producing hydroxamic acid compounds. In this synthesis, the hydroxamic acid group is introduced at the final step.

Ho et al., J. Org. Chem. 2005, 70, 4873-4875 discloses the addition of small amounts of solid KCN to solution and solid-phase esters in THF/MeOH/50% aqueous NH2OH increases the efficiency of their transformation to the corresponding hydroxamic acids. Also disclosed is that the analogous carboxylic acid is formed as a byproduct (see Table 1).

Park et al. Eur. J. Org. Chem. 2013, 1973-1978 discloses the synthesis of arylalkynecarboxylic acids from aryl bromides and alkynecarboxylic acids using a palladium catalyst, specifically, Pd(PPh₃)₄

DeVasher et al., J. Org. Chem., Vol. 69, No. 23, 2004, pp7919-7927 discloses the Sonogashira Coupling of aryl bromides with phenyl alkynes utilizing a palladium catalyst. This reference does not discloses Sonogashira Coupling of aryl bromides that have hydroxamic acid substituents.

However, there is no disclosure of the preparation of arylalkynehydroxamates from aryl bromides with hydroxamic acid substituents and arylalkynes using the Pd(PPh₃)₄ catalyst.

There is a need for methods to produce hydroxamic acid antibiotics with reduced formation of the corresponding carboxylic acid by product.

### SUMMARY OF THE INVENTION

The invention concerns a method of preparing a compound of Formula (I)

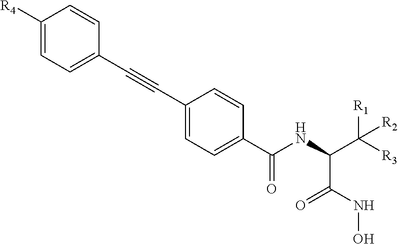

or salt thereof, comprising . . .
converting a compound of Formula (III)

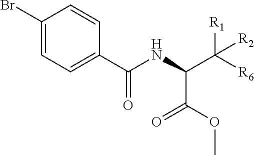

to a compound of Formula (IV)

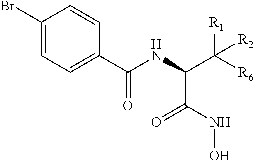

and coupling the compound of Formula (IV) with a compound of Formula (IX)

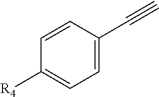

or a salt thereof
to yield the compound of Formula (I) with less than 2% of a compound of Formula (XI)

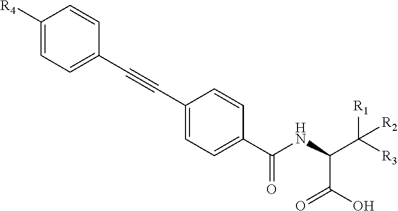

produced as a byproduct., wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

### DETAILED DESCRIPTION

Hydroxamic acid antibiotics are often produced by introducing the hydroxamic acid group in one of the final reaction steps by the conversion of the corresponding ester. These processes that produce hydroxamic acid derivatives often produce the corresponding carboxylic acid as a byproduct. Further tedious purification of the desired hydroxamic acid is required to remove the undesired carboxylic acid. The claimed invention is a process to produce hydroxamic acid derivatives with significantly reduced production of the undesired carboxylic acid byproduct.

Formation of the hydroxamic acid at the ultimate stage led to the isolation of the desired product in the presence of 3 to 8% carboxylic acid.

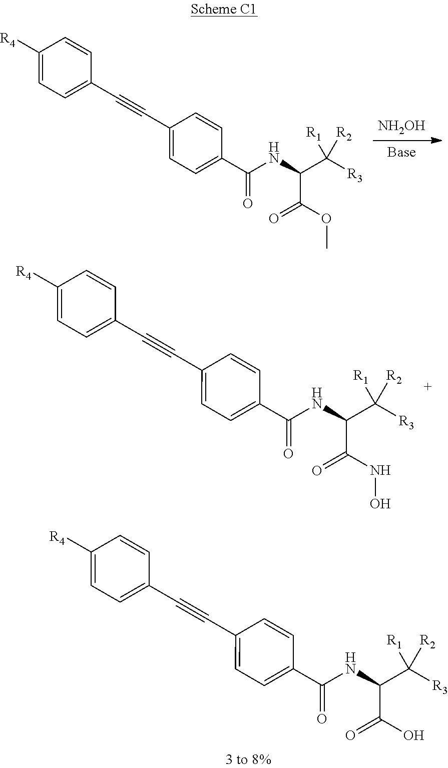

See comparative example 1 below.

Attempting the formation of the hydroxamic acid on the Boc-protected intermediate led to even larger amount of the carboxylic acid side product (14 to 20%).

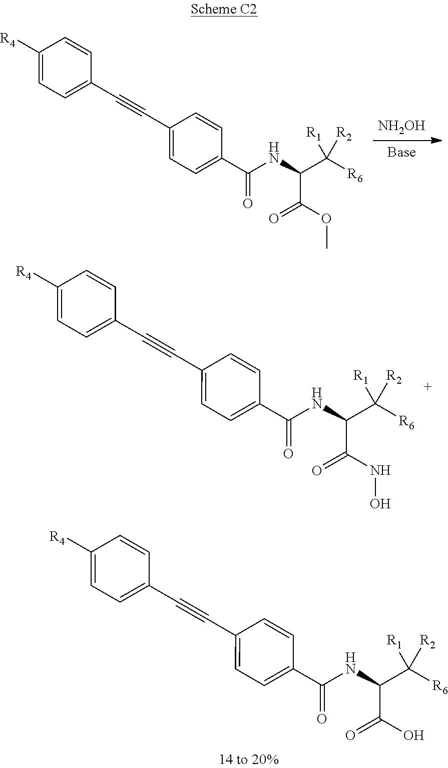

See comparative example 2 below.

Introduction of the hydroxamic acid early in the sequence of reaction steps reduces the amount of carboxylic acid produced along with the final product and enables easy purification by selective crystallization.

In contrast to the comparative examples, the claimed process leads to <2% carboxylic acid in a reproducible manner and allows for easy separation of this side product by selective crystallization of the desired hydroxamic acid derivative. Scheme 1 shows the overall inventive process with the introduction of the hydroxamate group in the second step of the reaction sequence.

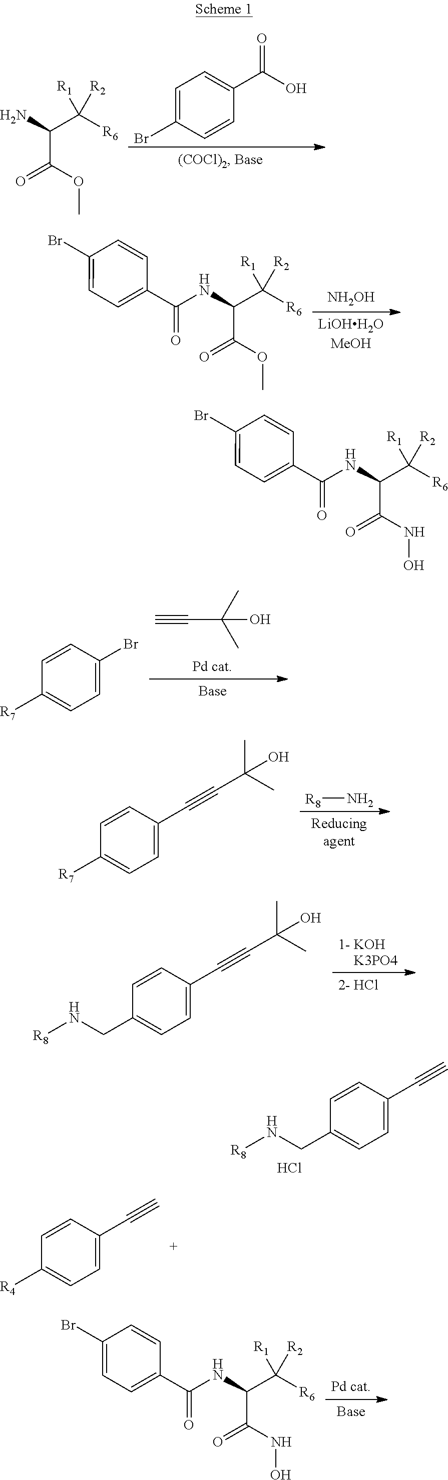

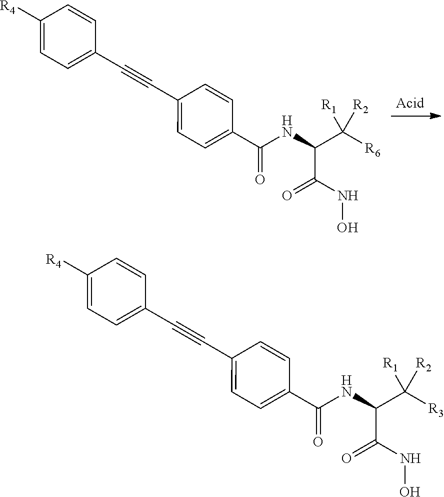

Scheme 2 shows the complete process when R₄ is cyclopropyl amine.

### Scheme 2

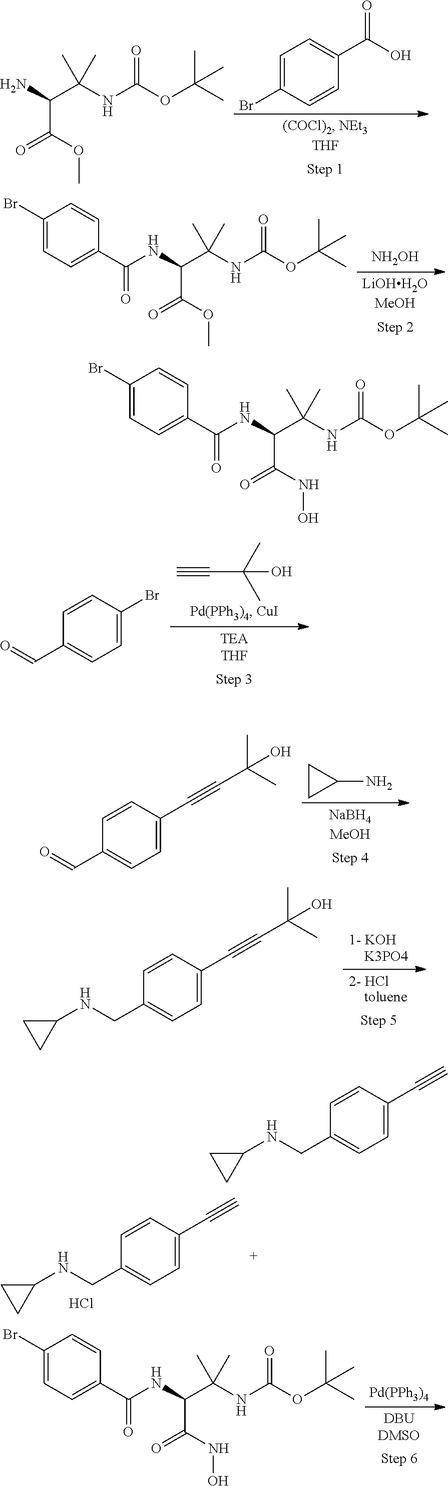

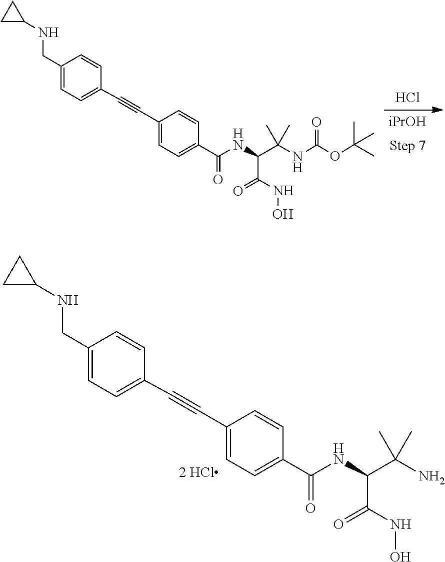 [SMILES: "iPrOH" ; CC(C)(C)OC(=O)NC(C)(C)[C@H](NC(=O)c1ccc(C#Cc2ccc(CNC3CC3)cc2)cc1)C(=O)NO ; Cl ; CC(C)(N)[C@H](NC(=O)c1ccc(C#Cc2ccc(CNC3CC3)cc2)cc1)C(=O)NO]

In an embodiment of the invention, the method of preparing a compound of Formula (I) comprises the step of reacting the compound of Formula (III)

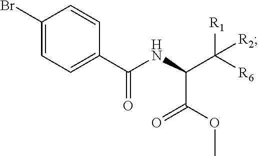

with hydroxylamine in the presence of lithium hydroxide to produce the compound of Formula (IV)

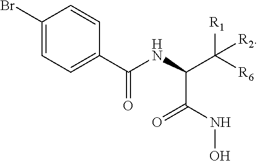

In an embodiment of the invention, the method of preparing a compound of Formula (I) comprises the step of coupling the compound of Formula (IV)

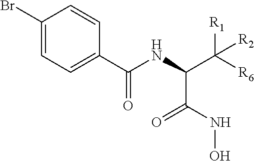

to the compound of Formula (IX)

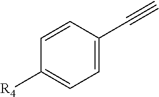

or a salt thereof in the presence of a palladium catalyst, a base and optionally copper iodide to yield a compound of Formula (X)

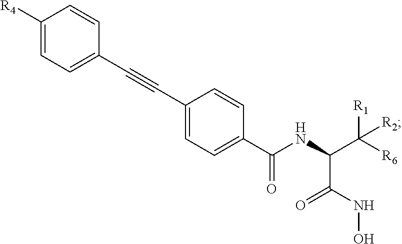

and then subsequently converting the compound of Formula (X) to
the compound of Formula (I) or a salt thereof.

In an embodiment of the invention, the method of preparing a compound of Formula (I) comprises the step of forming the compound of Formula (III) by reacting a compound of Formula (II)

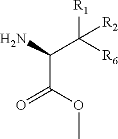

with 4 bromo benzoic acid to yield a compound of Formula (III).

In an embodiment of the invention, the method of preparing a compound of Formula (I) comprises the steps of
a) reacting a compound of Formula (II)

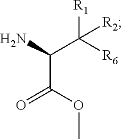

with 4 bromo benzoic acid to yield a compound of Formula (III)

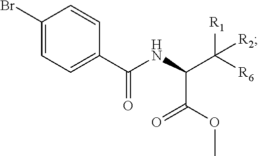

b) reacting the compound of Formula (III) with hydroxylamine in the presence of lithium hydroxide to produce a compound of Formula (IV)

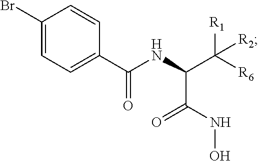

c) reacting the compound of Formula (IV) with a compound of Formula (IX)

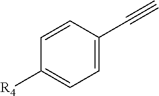

or a salt thereof
in the presence of a palladium catalyst, a base and optionally copper iodide to yield a compound of Formula (X)

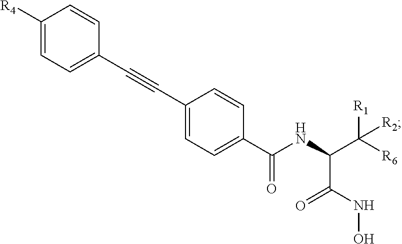

and
d) reacting the compound of Formula (X) with an acid to yield the compound of Formula (I) or a salt thereof
wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

In an embodiment of the invention, the palladium catalyst is a zero valent palladium catalyst. In an embodiment the palladium catalyst is Pd2(dba)₃; Pd(tBu3P)₂; Pd(Cy3P)₂ or Pd(PPh₃)₄., preferably Pd(PPh₃)₄.

In an embodiment of the invention, the palladium catalyst is Pd(PPh₃)₄.

In an embodiment of the invention, the compound of Formula I is selected from the group consisting of

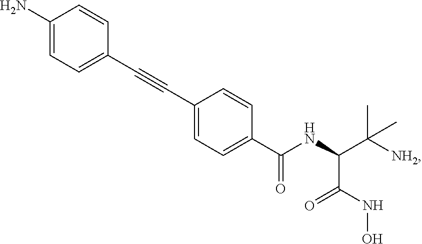 [SMILES: CC(C)(N)[C@H](NC(=O)c1ccc(C#Cc2ccc(N)cc2)cc1)C(=O)NO]

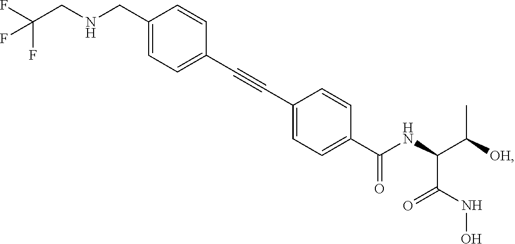 [SMILES: C[C@@H](O)[C@H](NC(=O)c1ccc(C#Cc2ccc(CNCC(F)(F)F)cc2)cc1)C(=O)NO]

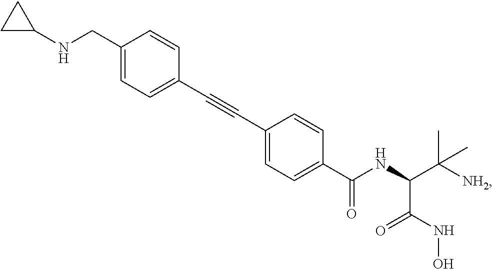 [SMILES: CC(C)(N)[C@H](NC(=O)c1ccc(C#Cc2ccc(CNC3CC3)cc2)cc1)C(=O)NO]

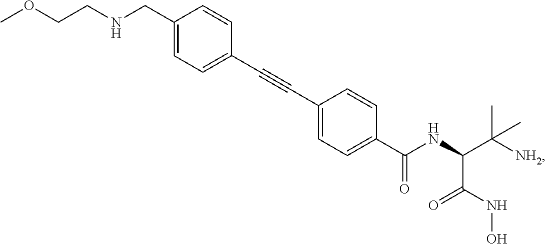 [SMILES: COCCNCc1ccc(C#Cc2ccc(C(=O)N[C@H](C(=O)NO)C(C)(C)N)cc2)cc1]

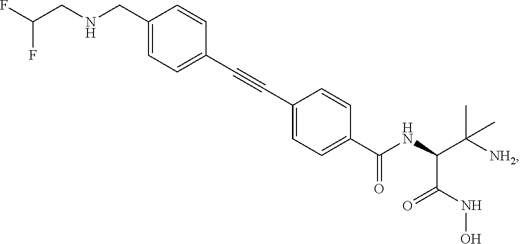 [SMILES: CC(C)(N)[C@H](NC(=O)c1ccc(C#Cc2ccc(CNCC(F)F)cc2)cc1)C(=O)NO]

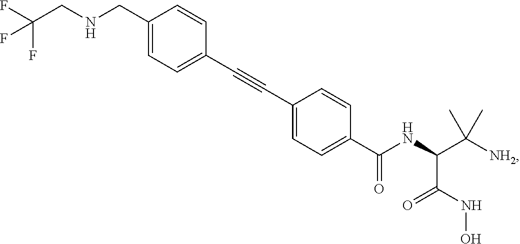 [SMILES: CC(C)(N)[C@H](NC(=O)c1ccc(C#Cc2ccc(CNCC(F)(F)F)cc2)cc1)C(=O)NO]

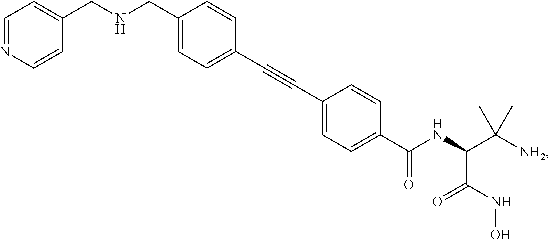 [SMILES: CC(C)(N)[C@H](NC(=O)c1ccc(C#Cc2ccc(CNCc3ccncc3)cc2)cc1)C(=O)NO]

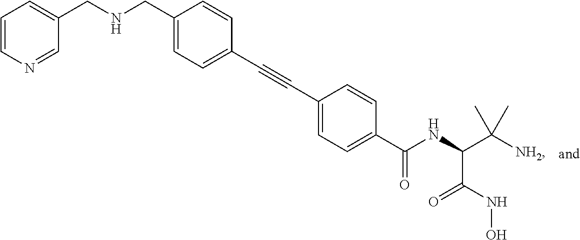 [SMILES: CC(C)(N)[C@H](NC(=O)c1ccc(C#Cc2ccc(CNCc3cccnc3)cc2)cc1)C(=O)NO]

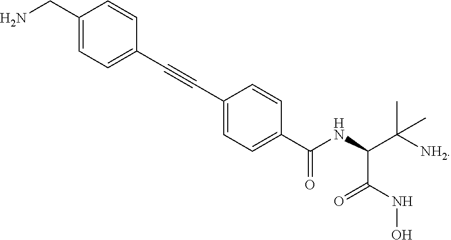 [SMILES: CC(C)(N)[C@H](NC(=O)c1ccc(C#Cc2ccc(CN)cc2)cc1)C(=O)NO]

In an embodiment of the invention, the compound of Formula (I) is a hydrobromide salt, a dihydrobromide salt, a hydrochloride salt or a dihydrochloride salt.

Another embodiment of the invention is a compound of Formula (X)

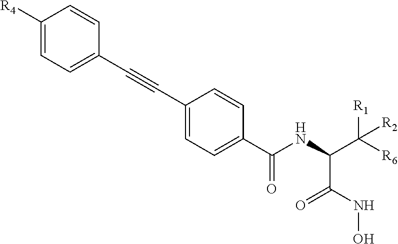

or a salt thereof
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃ or -pyridine; and
R⁶ is —NHC(O)OC(CH₃)₃ or —OC(CH₃)₃.

In an embodiment of the invention, the compound of Formula X is selected from the group consisting of

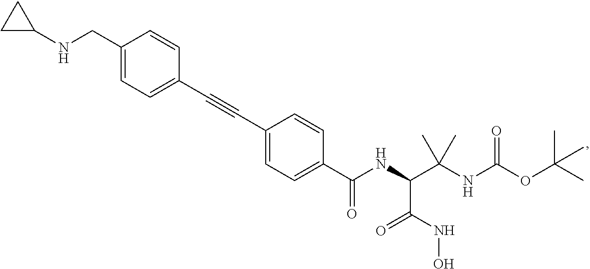 [SMILES: CC(C)(C)OC(=O)NC(C)(C)[C@H](NC(=O)c1ccc(C#Cc2ccc(CNC3CC3)cc2)cc1)C(=O)NO]

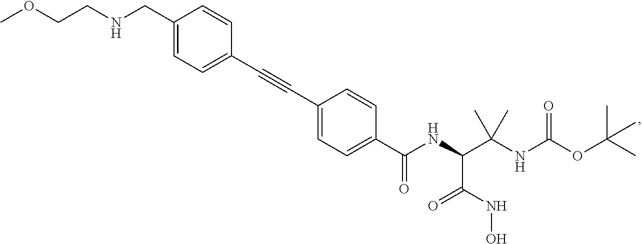 [SMILES: COCCNCc1ccc(C#Cc2ccc(C(=O)N[C@H](C(=O)NO)C(C)(C)NC(=O)OC(C)(C)C)cc2)cc1]

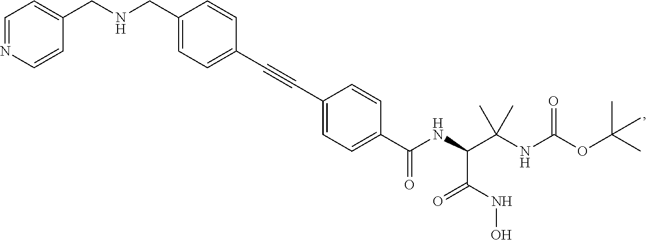 [SMILES: CC(C)(C)OC(=O)NC(C)(C)[C@H](NC(=O)c1ccc(C#Cc2ccc(CNCc3ccncc3)cc2)cc1)C(=O)NO]

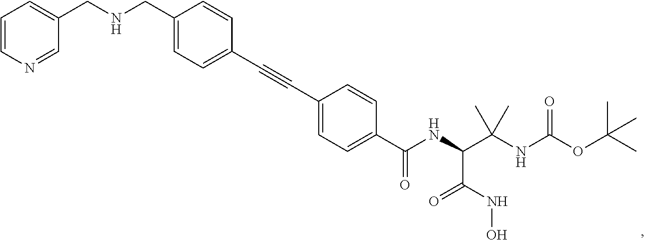 [SMILES: CC(C)(C)OC(=O)NC(C)(C)[C@H](NC(=O)c1ccc(C#Cc2ccc(CNCc3cccnc3)cc2)cc1)C(=O)NO]

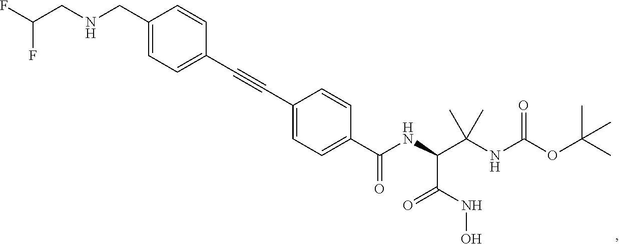 [SMILES: CC(C)(C)OC(=O)NC(C)(C)[C@H](NC(=O)c1ccc(C#Cc2ccc(CNCC(F)F)cc2)cc1)C(=O)NO]

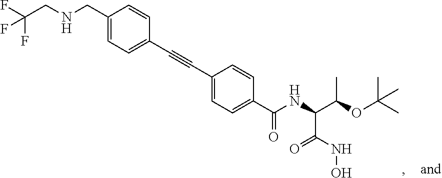 [SMILES: C[C@@H](OC(C)(C)C)[C@H](NC(=O)c1ccc(C#Cc2ccc(CNCC(F)(F)F)cc2)cc1)C(=O)NO]

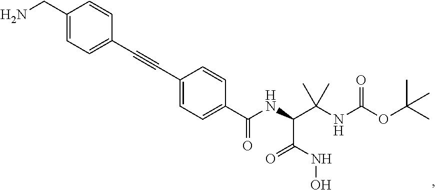 [SMILES: CC(C)(C)OC(=O)NC(C)(C)[C@H](NC(=O)c1ccc(C#Cc2ccc(CN)cc2)cc1)C(=O)NO]

In an embodiment of the invention, the compound is a hydrobromide salt, a dihydrobromide salt, a hydrochloride salt or a dihydrochloride salt.

Another embodiment of the invention is a method of preparing a compound of Formula (X)

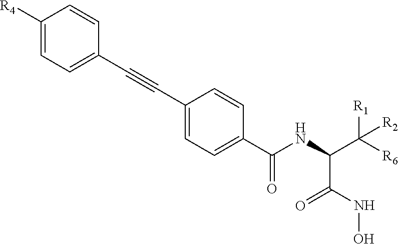

or a salt thereof
comprising reacting a compound of Formula (IX)

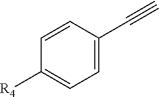

with a compound of Formula (IV)

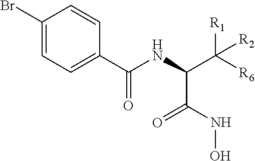

in the presence of a palladium catalyst, a base and optionally copper iodide to yield a compound of Formula (X) and optionally converting it to a salt,
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

In an embodiment of the invention, the palladium catalyst is a zero valent palladium catalyst. In an embodiment the palladium catalyst is Pd2(dba)₃; Pd(tBu3P)₂; Pd(Cy3P)₂ or Pd(PPh₃)₄., preferably Pd(PPh₃)₄.

In an embodiment of the invention, the palladium catalyst is Pd(PPh₃)₄.

Another embodiment of the invention is a compound of Formula (IX)

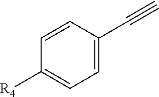

or a salt thereof, wherein
R⁴ is CH₂—NH—R⁵ or NH₂;
R⁵ is hydrogen, cyclopropyl or —CH₂— substituted by —CH₂OCH₃, —CHF₂, —CF₃, 3-pyridinyl or 4-pyridinyl.

In an embodiment of the invention, the compound of Formula IX is selected from the group consisting of

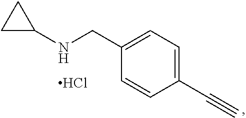

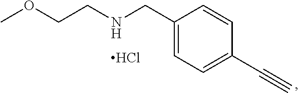

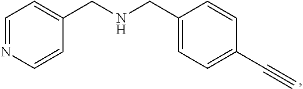 [SMILES: C#Cc1ccc(CNCc2ccncc2)cc1]

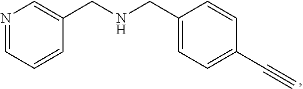 [SMILES: C#Cc1ccc(CNCc2cccnc2)cc1]

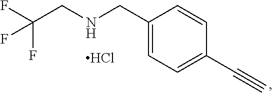

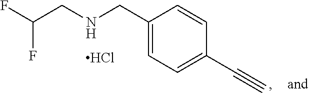

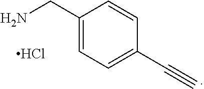

Another embodiment of the invention is a method of preparing a compound of Formula (IX)

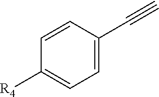

or a salt thereof comprising
a) reacting a compound of Formula (V)

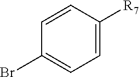

with HC≡C—C(CH₃)₂OH in the presence of a palladium catalyst, of a base and optionally of copper iodide to yield a compound of Formula (VI)

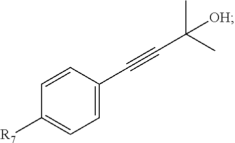

b) reacting the compound of Formula (VI) with a compound of Formula (VII)
R⁸—NH₂ in the presence of a reducing agent
to yield a compound of Formula (VIII)

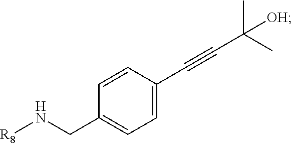

c) reacting either the compound of Formula (VI) or the compound of Formula (VIII) with KOH and K₃PO₄ to yield a compound of Formula (IX)
wherein
R⁴ is CH₂—NH—R⁵;
R⁵ is hydrogen, cyclopropyl or —CH₂— substituted by —CH₂OCH₃, —CHF₂, —CF₃, 3-pyridinyl or 4-pyridinyl;
R⁷ is C(O)H; and
R⁸ is cyclopropyl or —CH₂— substituted by —CH₂OCH₃, —CHF₂, —CF₃, 3-pyridinyl or 4-pyridinyl.

Another embodiment of the invention is a method of preparing a compound of Formula (IX)

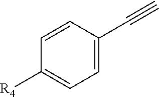

or a salt thereof comprising
a) reacting a compound of Formula (V)

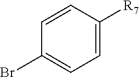

with HC≡C—C(CH₃)₂OH in the presence of a palladium catalyst, of a base and optionally of copper iodide to yield a compound of Formula (VI)

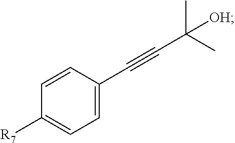

b) reacting either the compound of Formula (VI) with KOH and K₃PO₄ to yield a compound of Formula (IX), wherein R⁴ and R⁷ are NH₂.

Another embodiment of the invention is a compound of Formula (III)

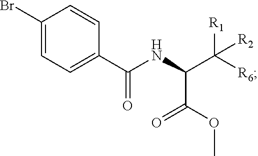

or a salt thereof
wherein R¹ and R² are each independently hydrogen or C₁ to C₄ alkyl and
R⁶ is —NHC(O)OC(CH₃)₃ or —OC(CH₃)₃.

In an embodiment of the invention, R¹ and R² are CH₃ and R⁶ is —NHC(O)OC(CH₃)₃.

In an embodiment of the invention, R¹ is CH₃, R² is hydrogen and R⁶ is —OC(CH₃)₃.

Another embodiment of the invention is a compound of Formula (IV)

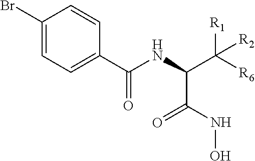

or a salt thereof
wherein R¹ and R² are each independently hydrogen or C₁ to C₄ alkyl and
R⁶ is —NHC(O)OC(CH₃)₃ or —OC(CH₃)₃.

In an embodiment of the invention, R¹ and R² are CH₃ and R⁶ is —NHC(O)OC(CH₃)₃.

In an embodiment of the invention, R¹ is CH₃, R² is hydrogen and R⁶ is —OC(CH₃)₃.

Another embodiment of the invention is a compound of Formula (VI)

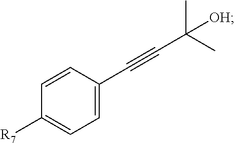

or a salt thereof,
wherein R⁷ is NH₂.

Another embodiment of the invention is a compound of Formula (VIII)

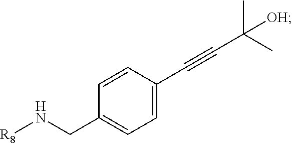

or a salt thereof,
wherein R⁸ is cyclopropyl or —CH₂— substituted by —CH₂OCH₃, —CHF₂, —CF₃, 3-pyridinyl or 4-pyridinyl.

In an embodiment of the invention, the compound of Formula VIII is selected from the group consisting of

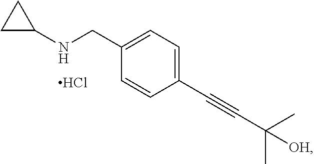 [SMILES: CC(C)(O)C#Cc1ccc(CNC2CC2)cc1 ; Cl]

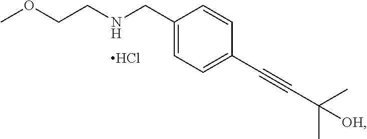 [SMILES: COCCNCc1ccc(C#CC(C)(C)O)cc1 ; Cl]

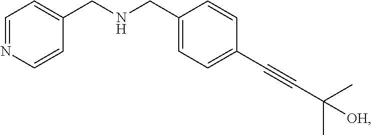 [SMILES: CC(C)(O)C#Cc1ccc(CNCc2ccncc2)cc1]

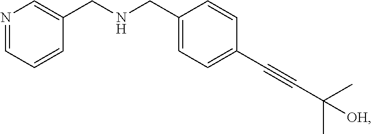 [SMILES: CC(C)(O)C#Cc1ccc(CNCc2cccnc2)cc1]

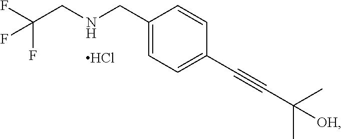 [SMILES: CC(C)(O)C#Cc1ccc(CNCC(F)(F)F)cc1 ; Cl]

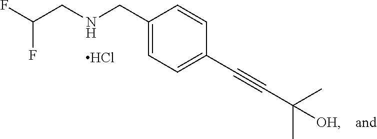

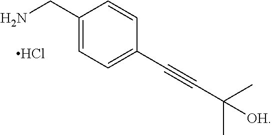

Hydroxamic acid—A hydroxamic acid is a class of organic compounds bearing the functional group RC(O)N(OH)R′, with R and R′ as organic residues and CO as a carbonyl group. They are amides (RC(O)NHR′) wherein the NH center has an OH substitution. They are often used as metal chelators.

Sonogashira coupling reaction: this coupling of terminal alkynes with aryl or vinyl halides is performed with a palladium catalyst and optionally a copper(I) cocatalyst (Rafael Chinchilla and Carmen Nájera Chem. Rev. 2007, 107, 3, 874-922 Publication Date: Feb. 17, 2007 https://doi.org/10.1021/cr050992x).

A reducing agent is a substance that tends to bring about reduction by being oxidized and losing electrons. Examples are sodium borohydride (NaBH₄), sodium cyanoborohydride and sodium triacetoxyborohydride (Na(CH₃CO₂)BH).

A zero valent palladium catalyst is a catalyst where the palladium metal atoms have a complete valence shell of electrons. Examples of such catalysts are Pd2(dba)₃; Pd(tBu3P)₂; Pd(Cy3P)₂ or Pd(PPh₃)₄.

### Additional Embodiments

Embodiment 1. A method of preparing a compound of Formula (I)

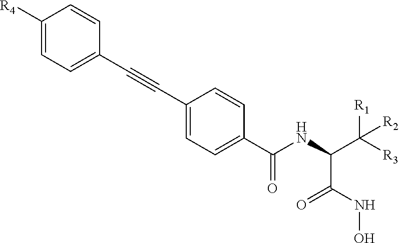

or salt thereof, comprising . . .
converting a compound of Formula (III)

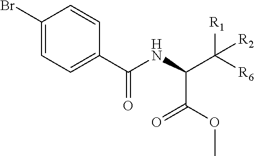

to a compound of Formula (IV)

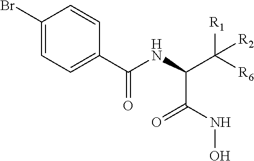

and coupling the compound of Formula (IV) with a compound of Formula (IX)

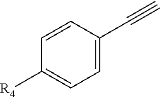

or a salt thereof to yield the compound of Formula (I) with less than 2% of a compound of Formula (XI)

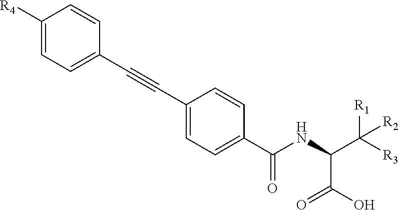

produced as a byproduct., wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

### Embodiment 2. The Method of Embodiment 1, Comprising the Step of Reacting the Compound of Formula (III)

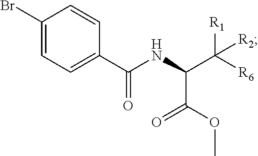

with hydroxylamine in the presence of lithium hydroxide to produce the compound of Formula

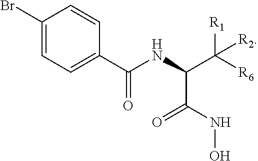

### Embodiment 3. The Method of any One of Embodiments 1 to 2 Comprising the Step of Coupling the Compound of Formula (IV)

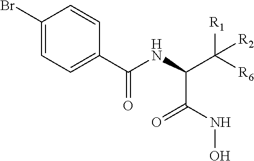

to the compound of Formula (IX)

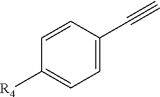

or a salt thereof in the presence of a palladium catalyst, a base and optionally copper iodide to yield a compound of Formula (X)

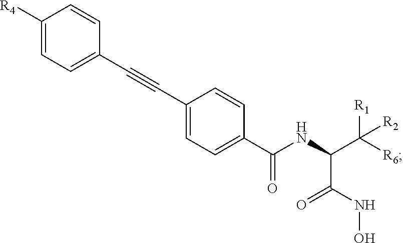

and then subsequently converting the compound of Formula (X) to
the compound of Formula (I) or a salt thereof.

### Embodiment 4. The Method of any One of Embodiments 1 to 3, Comprising the Step of Forming the Compound of Formula (III)

by reacting a compound of Formula (II)

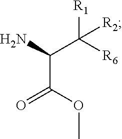

with 4 bromo benzoic acid to yield a compound of Formula (III).

### Embodiment 5. The Method of any One of Embodiments 1 to 4 Comprising the Steps of

a) reacting a compound of formula (ii)

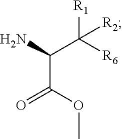

with 4 bromo benzoic acid to yield a compound of Formula (III)

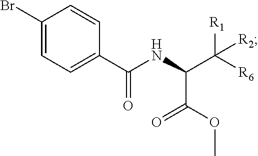

b) reacting the compound of Formula (III) with hydroxylamine in the presence of lithium hydroxide to produce a compound of Formula (IV)

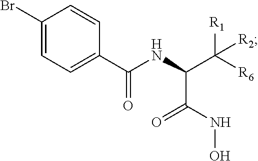

c) reacting the compound of Formula (IV) with a compound of Formula (IX)

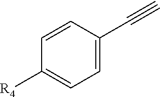

or a salt thereof in the presence of a palladium catalyst, a base and optionally copper iodide to yield a compound of Formula (X)

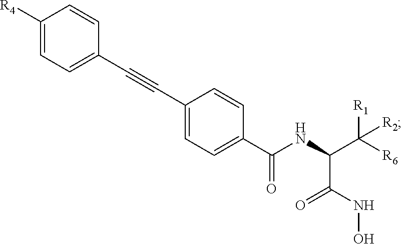

and
d) reacting the compound of Formula (X) with an acid to yield the compound of Formula (I) or a salt thereof
wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

Embodiment 6. The method of anyone of embodiments 3-5, wherein the palladium catalyst is Pd(PPh₃)₄.

Embodiment 7. The method of any one of embodiments 1-6, wherein the compound of Formula (I) is selected from the group consisting of

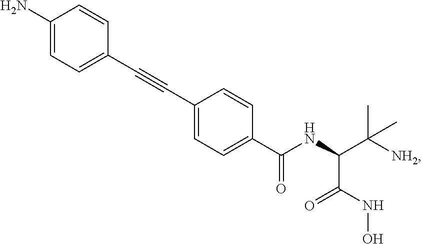 [SMILES: CC(C)(N)[C@H](NC(=O)c1ccc(C#Cc2ccc(N)cc2)cc1)C(=O)NO]

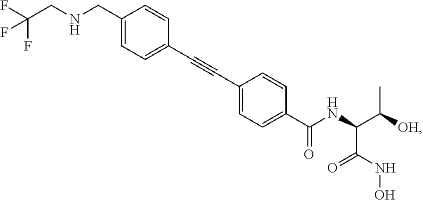 [SMILES: C[C@@H](O)[C@H](NC(=O)c1ccc(C#Cc2ccc(CNCC(F)(F)F)cc2)cc1)C(=O)NO]

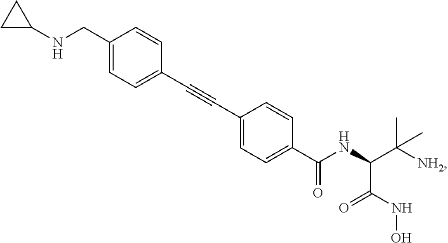 [SMILES: CC(C)(N)[C@H](NC(=O)c1ccc(C#Cc2ccc(CNC3CC3)cc2)cc1)C(=O)NO]

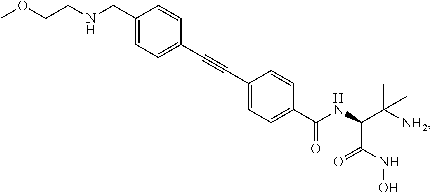 [SMILES: COCCNCc1ccc(C#Cc2ccc(C(=O)N[C@H](C(=O)NO)C(C)(C)N)cc2)cc1]

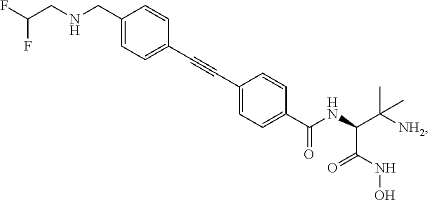 [SMILES: CC(C)(N)[C@H](NC(=O)c1ccc(C#Cc2ccc(CNCC(F)F)cc2)cc1)C(=O)NO]

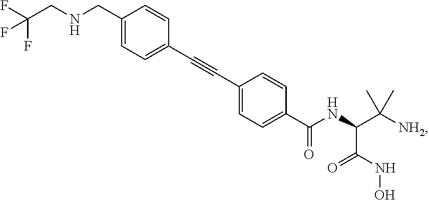 [SMILES: CC(C)(N)[C@H](NC(=O)c1ccc(C#Cc2ccc(CNCC(F)(F)F)cc2)cc1)C(=O)NO]

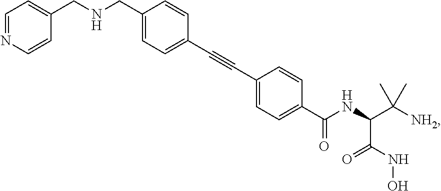 [SMILES: CC(C)(N)[C@H](NC(=O)c1ccc(C#Cc2ccc(CNCc3ccncc3)cc2)cc1)C(=O)NO]

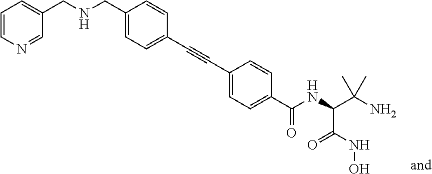 [SMILES: CC(C)(N)[C@H](NC(=O)c1ccc(C#Cc2ccc(CNCc3cccnc3)cc2)cc1)C(=O)NO]

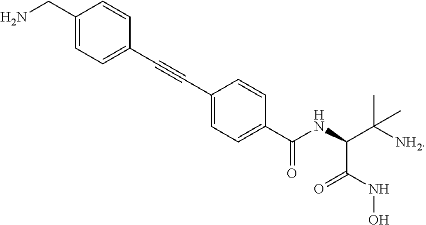 [SMILES: CC(C)(N)[C@H](NC(=O)c1ccc(C#Cc2ccc(CN)cc2)cc1)C(=O)NO]

Embodiment 8. The method of any one of embodiments 1-7, wherein the compound of Formula (I) is a hydrobromide salt, a dihydrobromide salt, a hydrochloride salt or a dihydrochloride salt.

Embodiment 9. A compound of Formula (X)

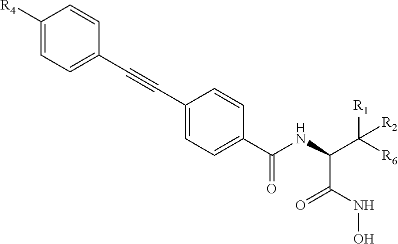

or a salt thereof
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃ or -3-pyridinyl or 4-pyridinyl; and
R⁶ is —NHC(O)OC(CH₃)₃ or —OC(CH₃)₃.

Embodiment 10. The compound of embodiment 9, wherein the compound of Formula X is selected from the group consisting of

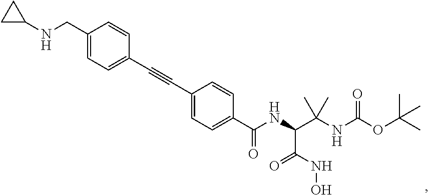 [SMILES: CC(C)(C)OC(=O)NC(C)(C)[C@H](NC(=O)c1ccc(C#Cc2ccc(CNC3CC3)cc2)cc1)C(=O)NO]

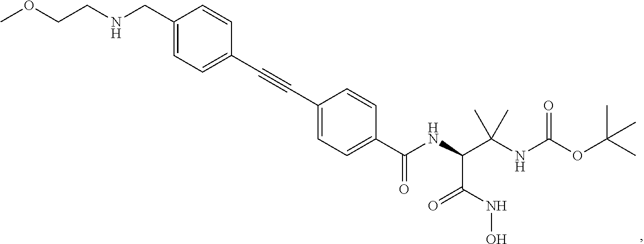 [SMILES: COCCNCc1ccc(C#Cc2ccc(C(=O)N[C@H](C(=O)NO)C(C)(C)NC(=O)OC(C)(C)C)cc2)cc1]

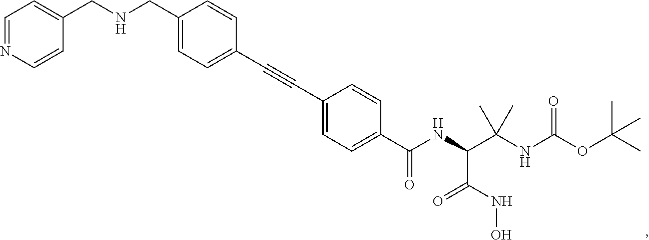 [SMILES: CC(C)(C)OC(=O)NC(C)(C)[C@H](NC(=O)c1ccc(C#Cc2ccc(CNCc3ccncc3)cc2)cc1)C(=O)NO]

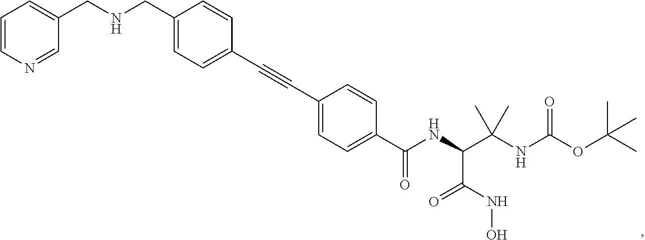 [SMILES: CC(C)(C)OC(=O)NC(C)(C)[C@H](NC(=O)c1ccc(C#Cc2ccc(CNCc3cccnc3)cc2)cc1)C(=O)NO]

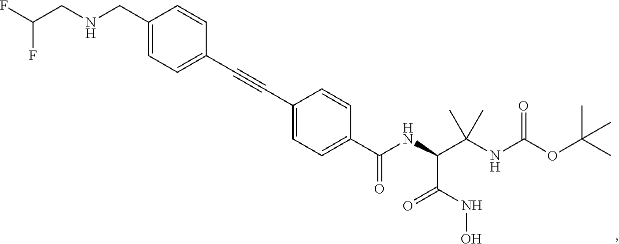 [SMILES: CC(C)(C)OC(=O)NC(C)(C)[C@H](NC(=O)c1ccc(C#Cc2ccc(CNCC(F)F)cc2)cc1)C(=O)NO]

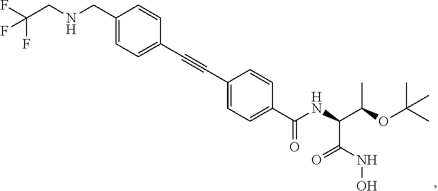 [SMILES: C[C@@H](OC(C)(C)C)[C@H](NC(=O)c1ccc(C#Cc2ccc(CNCC(F)(F)F)cc2)cc1)C(=O)NO]

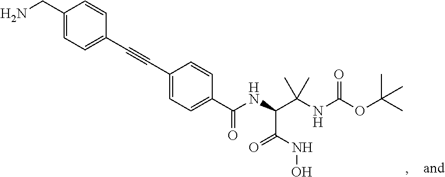 [SMILES: CC(C)(C)OC(=O)NC(C)(C)[C@H](NC(=O)c1ccc(C#Cc2ccc(CN)cc2)cc1)C(=O)NO]

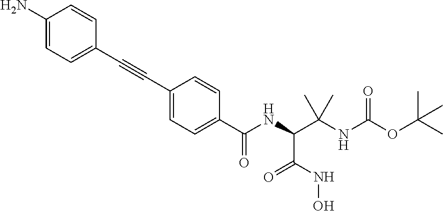 [SMILES: CC(C)(C)OC(=O)NC(C)(C)[C@H](NC(=O)c1ccc(C#Cc2ccc(N)cc2)cc1)C(=O)NO]

or salt thereof

Embodiment 11. The compound of anyone of embodiments 9-10, wherein the compound is a hydrobromide salt, a dihydrobromide salt, a hydrochloride salt or a dihydrochloride salt.

Embodiment 12. A method of preparing a compound of any one of embodiments 9 to 11 comprising reacting a compound of Formula (IX)

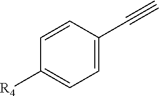

or salt thereof
with a compound of Formula (IV)

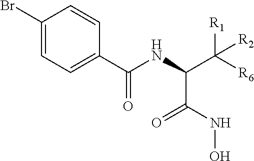

in the presence of a palladium catalyst, a base and optionally copper iodide to yield a compound of Formula (X) and optionally converting it to a salt
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

Embodiment 13. The method of embodiment 12, wherein the palladium catalyst is Pd(PPh₃)₄.

### Embodiment 14. A Compound of Formula (IX)

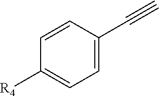

or a salt thereof, wherein
R⁴ is CH₂—NH—R⁵ or NH₂;
R⁵ is hydrogen, cyclopropyl or —CH₂— substituted by —CH₂OCH₃, —CHF₂, —CF₃, 3-pyridinyl or 4-pyridinyl.

Embodiment 15. The compound of embodiment 14, wherein the compound of Formula IX is selected from the group consisting of

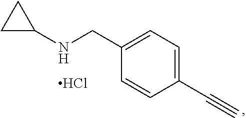

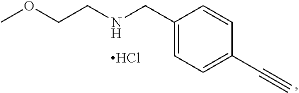

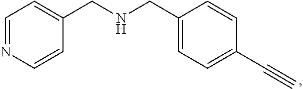 [SMILES: C#Cc1ccc(CNCc2ccncc2)cc1]

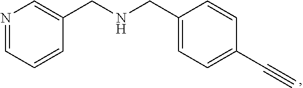 [SMILES: C#Cc1ccc(CNCc2cccnc2)cc1]

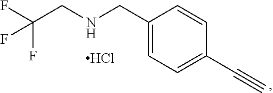

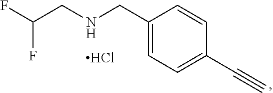

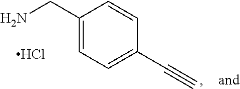

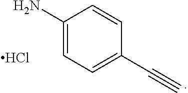

Embodiment 16. A method of preparing the compound of any one of embodiments 14 or 15 or a salt thereof comprising
a) reacting a compound of Formula (V)

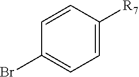

with HC≡C—C(CH₃)₂OH in the presence of a palladium catalyst, of a base and optionally of copper iodide to yield a compound of Formula (VI)

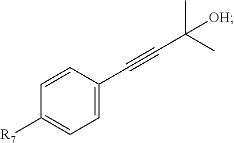

b) reacting the compound of Formula (VI) with a compound of Formula (VII)
R⁸—NH₂ in the presence of a reducing agent
to yield a compound of Formula (VIII)

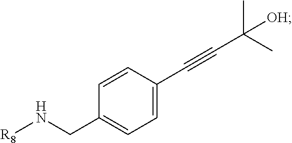

c) reacting either the compound of Formula (VI) or the compound of Formula (VIII) with KOH and K₃PO₄ to yield a compound of Formula (IX)
wherein
R⁴ is CH₂—NH—R⁵;
R⁵ is hydrogen, cyclopropyl or —CH₂— substituted by —CH₂OCH₃, —CHF₂, —CF₃, 3-pyridinyl or 4-pyridinyl;
R⁷ is C(O)H; and
R⁷ is cyclopropyl or —CH₂— substituted by —CH₂OCH₃, —CHF₂, —CF₃, 3-pyridinyl or 4-pyridinyl.

Embodiment 17. A method of preparing the compound of any one of embodiments 14 or 15

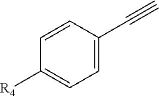

or a salt thereof comprising
a) reacting a compound of Formula (V)

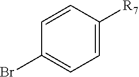

with HC≡C—C(CH₃)₂OH in the presence of a palladium catalyst, of a base and optionally of copper iodide to yield a compound of Formula (VI)

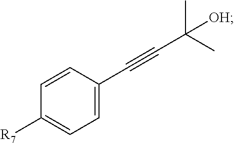

b) reacting either the compound of Formula (VI) with KOH and K₃PO₄ to yield the compound of Formula (IX),
wherein R⁴ and R⁷ are NH₂.

Embodiment 18. A compound of Formula (III)

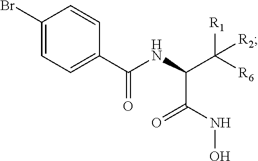

or a salt thereof
wherein R¹ and R² are each independently hydrogen or C₁ to C₄ alkyl and
R⁶ is —NHC(O)OC(CH₃)₃ or —OC(CH₃)₃.

Embodiment 19. The compound of embodiment 18, wherein R¹ and R² are CH₃ and R⁶ is —NHC(O)OC(CH₃)₃.

Embodiment 20. The compound of embodiment 18, wherein R¹ is CH₃, R² is hydrogen and R⁶ is —OC(CH₃)₃.

### Embodiment 21. A Compound of Formula (IV)

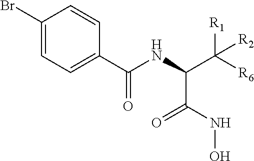

or a salt thereof
wherein R¹ and R² are each independently hydrogen or C₁ to C₄ alkyl and
R⁶ is —NHC(O)OC(CH₃)₃ or —OC(CH₃)₃.

Embodiment 22. The compound of embodiment 21, wherein R¹ and R² are CH₃ and R⁶ is —NHC(O)OC(CH₃)₃.

Embodiment 23. The compound of embodiment 21, wherein R¹ is CH₃, R² is hydrogen and R⁶ is —OC(CH₃)₃.

### Embodiment 24. A Compound of Formula (VI)

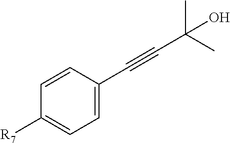

or a salt thereof,
wherein R⁷ is NH₂.

### Embodiment 25. A Compound of Formula (VIII)

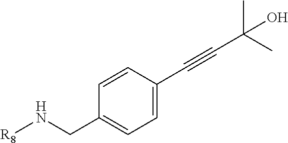

or a salt thereof,
wherein R⁸ is cyclopropyl or —CH₂— substituted by —CH₂OCH₃, —CHF₂, —CF₃, 3-pyridinyl or 4-pyridinyl.

Embodiment 26. The compound of embodiment 25, wherein the compound of Formula VIII is selected from the group consisting of

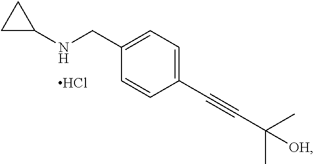 [SMILES: CC(C)(O)C#Cc1ccc(CNC2CC2)cc1 ; Cl]

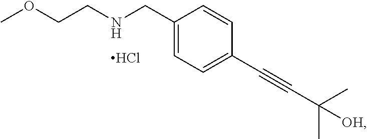 [SMILES: COCCNCc1ccc(C#CC(C)(C)O)cc1 ; Cl]

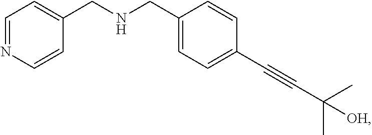 [SMILES: CC(C)(O)C#Cc1ccc(CNCc2ccncc2)cc1]

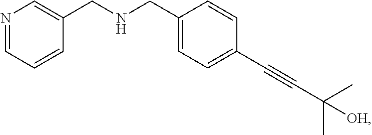 [SMILES: CC(C)(O)C#Cc1ccc(CNCc2cccnc2)cc1]

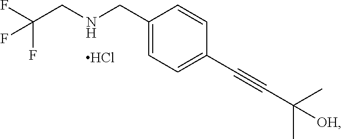 [SMILES: CC(C)(O)C#Cc1ccc(CNCC(F)(F)F)cc1 ; Cl]

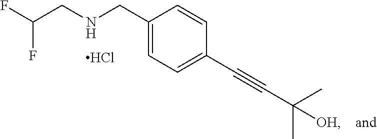

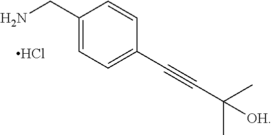

### EXAMPLES

### HPLC Methods:

### Method A

Agilent Technologies UHPLC/MSD 6130 B Series 1290 composed of
Binary pump G7120A included degasser
Well plate sampler G4226A
Column oven G1316B
Diode array detector G4212A
Mass detector G6130B Quadrupole LC/MS with ESI-source
Column: Waters XP, 2.1×50 mm Xbridge BEH C18 2.5μ, T=40° C.;
Eluents: A: acetonitrile with 0.05% (vol./vol.) formic acid.
B: water with 0.05% formic acid (vol./vol.);
Flow: 0.8 mL/min;
Gradient: from 2 to 100% eluent A 1.2 min, 0.5 min 100% eluent A;
Run time: 2.2 min;
Detection: ESI/MS, positive and negative ions scan: 100-1000 m/z;
UV at 254 and 210 nm;

### Method B

Agilent Technologies UHPLC/MS 1260 Series composed of:
Binary pump G7120A included degasser
Well plate sampler G4226A
Column oven G7116B
Diode array detector G7117B
Mass detector G6150B Quadrupole LC/MS with ESI-jetstream-source
Column: Waters XP, 2.1×50 mm Xbridge BEH C18 2.5μ, T=40° C.;
Eluents: A: acetonitrile with 0.05% (vol./vol.) formic acid.
B: water with 0.05% formic acid (vol./vol.);
Flow: 0.8 mL/min;
Gradient: from 2 to 100% eluent A 1.2 min, 0.5 min 100% eluent A;
Run time: 2.2 min;
Detection: ESIMS, positive and negative ions scan: 100-1000 m/z;
UV at 254 and 210 nm;

### Method C

Agilent Technologies UHPLC/MS 1260 Series composed of:
Binary pump G4220A included degasser
Well plate sampler G4226A
Column oven G7116B
Diode array detector G4212A
Mass detector G6130B Quadrupole LC/MS with ESI/APCI-multi mode source
Column: Waters XP, 2.1×50 mm Xbridge BEH C18 2.5μ, T=40° C.;
Eluents: A: acetonitrile with 0.05% (vol./vol.) formic acid.
B: water with 0.05% formic acid (vol./vol.);
Flow: 0.8 mL/min;
Gradient: from 2 to 100% eluent A 1.2 min, 0.5 min 100% eluent A;
Run time: 2.2 min;
Detection: ESIMS, positive and negative ions scan: 100-1000 m/z;
UV at 254 and 210 nm;

### Comparative example 1: Preparation of (S)-N-(3-amino-1-(hydroxyamino)-3-methyl-1-oxobutan-2-yl)-4-((4-((cyclopropylamino)methyl)phenyl)ethynyl)benzamide

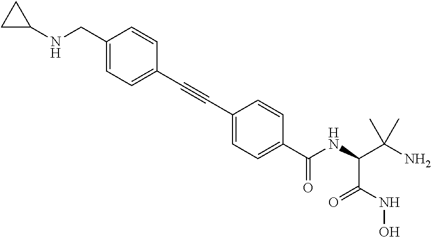 [SMILES: CC(C)(N)[C@H](NC(=O)c1ccc(C#Cc2ccc(CNC3CC3)cc2)cc1)C(=O)NO]

Methyl (S)-3-amino-2-(4-((4-((cyclopropylamino)methyl)phenyl)ethynyl)benzamido)-3-methylbutanoate hydrochloride (1 g, 2.19 mmol) was dissolved in methanol (10 mL). The resulting solution was cooled to 0° C. and aqueous 50% hydroxylamine (1.61 mL, 26.3 mmol) was added dropwise, while keeping the temperature below 2° C. Subsequently lithium hydroxide monohydrate (0.28 g, 6.58 mmol) was added in portions while maintaining the temperature below 2° C. After 2 h reaction time, the reaction mixture was allowed to warm to ambient temperature and was further stirred for 18 h. The reaction mixture was diluted with water (40 mL) and the volatiles were removed by evaporation under reduced pressure. The pH of the remaining aqueous layer was adjusted to pH 8.5 by the addition of aqueous 1M hydrochloric acid and the aqueous layer was extracted with a 2:1 mixture of dichloromethane and 2-propanol (2×50 mL). The organic layer was washed with brine (20 mL) and was concentrated under reduced pressure to afford the desired product as a yellow solid (775 mg, 1.70 mmol) in the presence of 8 area % of the corresponding carboxylic acid.

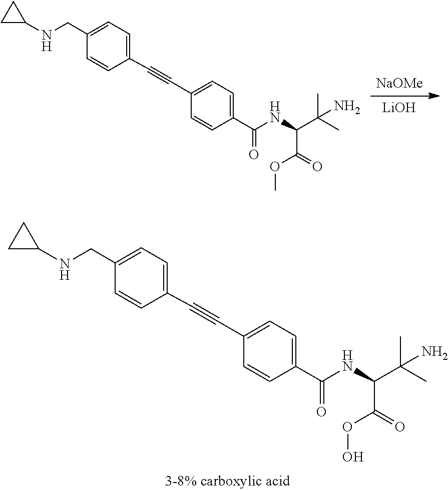

LC/MS (Method A): Rt=0.53 min, m/z 419 (desired product) and Rt=0.57 min, m/z 404 (carboxylic acid) Comparative example 2: Preparation of tert-butyl (S)-(3-(4-((4-((cyclopropylamino)methyl)phenyl)ethynyl)benzamido)-4-(hydroxyamino)-2-methyl-4-oxobutan-2-yl)carbamate

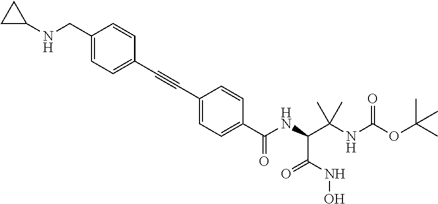 [SMILES: CC(C)(C)OC(=O)NC(C)(C)[C@H](NC(=O)c1ccc(C#Cc2ccc(CNC3CC3)cc2)cc1)C(=O)NO]

Methyl (S)-3-((tert-butoxycarbonyl)amino)-2-(4-((4-((cyclopropylamino)methyl)phenyl) ethynyl)benzamido)-3-methylbutanoate (0.50 g, 0.96 mmol) was dissolved in methanol (5 mL). The resulting solution was cooled to −5° C. and aqueous 50% hydroxylamine (0.71 mL, 11.55 mmol) was added dropwise while maintaining the temperature below −2° C. Lithium hydroxide monohydrate (0.081 g, 1.92 mmol) was added and after 15 min reaction time, the reaction mixture was allowed to reach room temperature. After 19 h reaction time at room temperature, water (5 mL) is added and the volatiles were removed by evaporation under reduced pressure. The pH of the aqueous residue was adjusted to pH 9.5 by the addition of aqueous 1N hydrochloric acid and the aqueous layer was extracted with methyl tert-butyl ether (2×20 mL). The combined organic layers were dried over sodium sulphate, filtered, and concentrated under reduced pressure to afford the desired product as a light yellow solid (500 mg, 0.86 mmol) in the presence of 14 area % of the corresponding carboxylic acid.

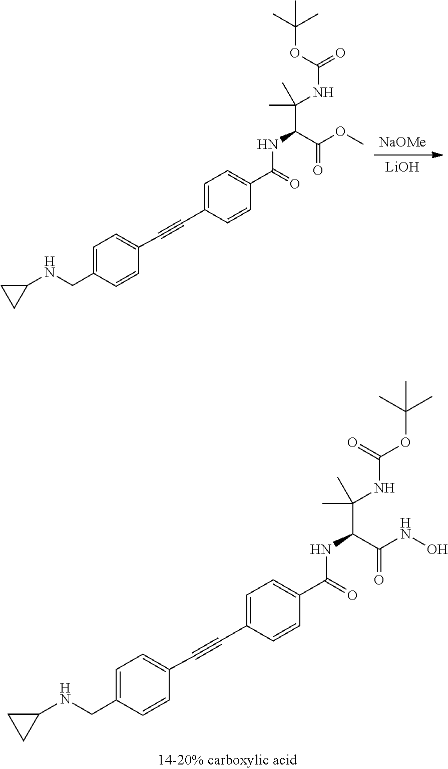

LC/MS (Method A): Rt=0.82 min, m/z 519 (desired product) and Rt=0.90 min, m/z 504 (carboxylic acid)

### Preparation of Compounds of Formula (IX)

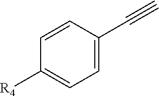

The compounds of Formula (IX) can be prepared following the synthetic method disclosed in example 1. The compounds of Formula (IX) can conveniently be isolated either as free bases or as hydrochloride salts as disclosed in examples 1 to 6.

### Example 1: Preparation of N-(4-ethynylbenzyl)cyclopropanamine hydrochloride

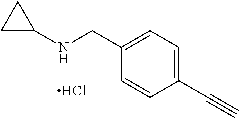

### Step 1: Preparation of 4-(3-hydroxy-3-methylbut-1-yn-1-yl)benzaldehyde

A jacketed glass reactor (2 L) was charged with a substituted 4-bromo benzaldehyde (100 g, 540 mmol), tetrakis(triphenylphosphine)palladium(0) (3.12 g, 2.70 mmol) and copper(I) iodide (1.029 g, 5.40 mmol) and was flushed with nitrogen. Dry tetrahydrofuran was added (1 L) and the resulting mixture was stirred at ambient temperature. After 15 min, triethylamine (151 mL, 1081 mmol) and 2-methylbut-3-yn-2-ol (53.9 ml, 540 mmol) were sequentially added. The temperature of the reaction mixture increased upon addition of the alcohol. However, the temperature of the mixture was maintained below 30° C. After completion of the addition the reaction mixture was brought to reflux (about 62° C.). After 70 minutes reaction time, the temperature of the reaction mixture was lowered to 15° C. and the mixture was filtered over a frit funnel filled with thin layers of cellulose and celite. The filter cake was washed with ethyl acetate (about 750 mL) and was dried under reduced pressure at 40° C. to afford the desired compound. Reduction of the volume of the mother liquor to about 2 volumes, followed by dilution of the concentrate with ethyl acetate (750 mL) and extraction of the organic layer with water (2×300 mL) allowed after concentration of the organic layer the isolation of a second crop of desired product. The combined crops of desired product (105 g, 530 mmol) were engaged in the next step without further purification.

¹H NMR (300 MHz, d3-Dimethylsulfoxide) δ (ppm): 10.0 (1H, s), 7.90 (2H, J=8.3 Hz, bd), 7.60 (2H, J=8.3 Hz, bd), 5.59 (1H, s), 1.49 (6H, s).

LC/MS (Method B): Rt=0.95 min, m/z 230

### Step 2: Preparation of 4-(4-((cyclopropylamino)methyl)phenyl)-2-methylbut-3-yn-2-ol

A jacketed glass reactor (5 L) was charged with methanol (1750 mL), 4-(3-hydroxy-3-methylbut-1-yn-1-yl)benzaldehyde (105 g, 530 mmol) and cyclopropylamine (44.7 mL, 636 mmol) and the resulting mixture was stirred at ambient temperature for 3 h. The temperature of the reaction mixture was then lowered to 5° C. prior to the portioned addition of sodium borohydride (14.2 g, 371 mmol) in order to maintain the temperature of the reaction mixture between 5 and 10° C. After completion of the addition, the reaction mixture was stirred between 5 and 10° C. for 1 h and water (500 mL) was added while maintaining the temperature below 10° C. The reaction mixture was then filtered over a frit funnel filled with thin layers of cellulose and celite and the reactor was rinsed twice with a mixture of water (60 mL) and methanol (210 mL) which were used to wash the wet cake. Aqueous hydrochloric acid was added to the filtrate while maintaining the temperature below 10° C. until pH 1 was reached. The resulting mixture was stirred for 30 min and the phases are allowed to settle. The aqueous layer was collected, was diluted with toluene (1 L) and the pH was adjusted to pH 8.5 be the addition of aqueous sodium hydroxide. After 15 min stirring the phases were allowed to settle, and the organic layer was collected. Sodium chloride (150 g) was added to the aqueous layer and toluene (400 mL) was added. The resulting mixture was stirred for 30 min and after separation of the phases, the second organic layer was combined with the first one. The combined organic layers were engaged in the next step.

¹H NMR (300 MHz, d3-Dimethylsulfoxide) δ (ppm): 8.20 (1H, s), 7.33 (4H, s), 3.78 (2H, s), 2.14-2.07 (1H, m), 1.45 (6H, s), 0.42-0.37 (2H, m), 0.34-0.29 (2H, m).

LC/MS (Method B): Rt=0.95 min, m/z 230

### Step 3: Preparation of N-(4-ethynylbenzyl)cyclopropanamine hydrochloride

A solution of 4-(4-((cyclopropylamino)methyl)phenyl)-2-methylbut-3-yn-2-ol (121 g, 527 mmol) in toluene (2500 mL) was charged in a jacketed glass reactor (5 L) and potassium hydroxide (30 g, 535 mmol) and potassium phosphate tribasic (112 g, 528 mmol) are added. The temperature was set to 85° C. and the reaction mixture was stirred at this temperature for 135 min. Heating was then stopped and the reaction mixture was allowed to reach room temperature before water (750 mL) was added. The resulting mixture was stirred for 30 min and the phases are allowed to settle. The aqueous layer was discarded and aqueous 1N sodium hydroxide (500 mL) was added. After stirring for 15 min, the phases are allowed to settle and the aqueous phase was discarded. The organic layer was then washed again with water (500 mL) and was diluted with toluene (500 mL). A 5N solution of hydrochloric acid in 2-propanol (160 mL) was added under stirring while maintaining the temperature below 25° C. The reaction mixture was stirred for 16 h at this temperature. The obtained suspension was filtered and the wet cake was rinsed with ethyl acetate (500 mL) and with methyl tert-butylether (500 mL). The rinsed wet cake was finally dried under reduced pressure at 35° C. to afford the desired product (79.5 g, 380 mmol, 72% yield).

¹H NMR (300 MHz, d3-Dimethylsulfoxide) δ (ppm): 9.71 (2H, s), 7.61 (2H, J=8.4 Hz, d), 7.53 (2H, J=8.4 Hz, d), 4.30 (1H, s), 4.21 (2H, s), 2.66-2.56 (1H, m), 0.95-0.90 (2H, m), 0.74-0.67 (2H, m).

LC/MS (Method B): Rt=1.01 min, m/z 172

### Example 2: Preparation of N-(4-ethynylbenzyl)-2-methoxyethan-1-amine hydrochloride

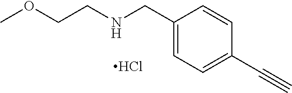

N-(4-ethynylbenzyl)-2-methoxyethan-1-amine hydrochloride was prepared following the general method described in example 1 using the appropriate amine. The desired product was isolated as a white solid (82% yield).

¹H NMR (300 MHz, d4-Methanol) δ (ppm): 7.48-7.44 (2H, m), 7.41-7.38 (2H, m), 4.14 (2H, s), 3.57-3.54 (2H, m), 3.53 (1H, s), 3.31 (3H, s), 3.15-3.10 (2H, m).

LC/MS (Method A): Rt=0.57 min, m/z 190

### Example 3: Preparation of N-(4-ethynylbenzyl)-1-(pyridin-4-yl)methanamine

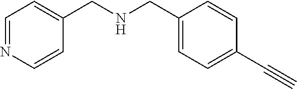 [SMILES: C#Cc1ccc(CNCc2ccncc2)cc1]

The preparation of N-(4-ethynylbenzyl)-1-(pyridin-4-yl)methanamine was achieved by following steps 1 and 2 of the general method described in example 1 to produce 2-methyl-4-(4-(((pyridin-4-ylmethyl)amino)methyl)phenyl)but-3-yn-2-ol. Step 3 was performed as follows.

### Step 3: Preparation of N-(4-ethynylbenzyl)-1-(pyridin-4-yl)methanamine

A solution of 2-methyl-4-(4-(((pyridin-4-ylmethyl)amino)methyl)phenyl)but-3-yn-2-ol (60 g, 213 mmol) in toluene (1300 mL) was charged in a jacketed glass reactor (3 L) and potassium hydroxide (12 g, 213 mmol) and potassium phosphate tribasic (45 g, 213 mmol) were added. The temperature was set to 100° C. and the reaction mixture was stirred at this temperature for 4 h. Heating was then stopped, and the reaction mixture was allowed to reach room temperature before water (350 mL) was added. The resulting mixture was stirred for 30 min and is filtered. The phases were allowed to settle, and the aqueous layer was discarded. The organic layer was sequentially washed with aqueous 1N sodium hydroxide (250 mL), with water (2×150 mL) and with brine (100 mL). Concentration of the organic layer under reduced pressure finally afforded the desired product (37.9 g, 157 mmol, 74% yield)

¹H NMR (300 MHz, d3-Dimethylsulfoxide) δ (ppm): 8.49-8.47 (2H, m), 7.44-7.34 (6H, m), 4.14 (1H, s), 3.68 (4H, s), 2.90 (1H, s).

LC/MS (Method A): Rt=0.49 min, m/z 223

### Example 4: Preparation of N-(4-ethynylbenzyl)-1-(pyridin-3-yl)methanamine

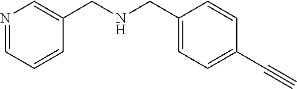 [SMILES: C#Cc1ccc(CNCc2cccnc2)cc1]

The preparation of N-(4-ethynylbenzyl)-1-(pyridin-3-yl)methanamine was achieved by following steps 1 and 2 of the general method described in example 1 to produce 2-methyl-4-(4-(((pyridin-3-ylmethyl)amino)methyl)phenyl)but-3-yn-2-ol. Step 3 was performed as follows.

### Step 3: Preparation of N-(4-ethynylbenzyl)-1-(pyridin-3-yl)methanamine

A solution of 2-methyl-4-(4-(((pyridin-3-ylmethyl)amino)methyl)phenyl)but-3-yn-2-ol (60 g, 213 mmol) in toluene (1.2 L) was charged in a jacketed glass reactor (3 L) and potassium hydroxide (12 g, 213 mmol) and potassium phosphate tribasic (45 g, 213 mmol) were added. The temperature was set to reflux and the reaction mixture was stirred at this temperature for 3.5 h. Heating was then stopped and the reaction mixture was allowed to reach room temperature before water (350 mL) was added. The resulting mixture was stirred for 30 min and filtered. The phases were allowed to settle and the aqueous layer was discarded. The organic layer was sequentially washed with aqueous 1N sodium hydroxide (250 mL), with water (2×125 mL) and with brine (100 mL). Concentration of the organic layer under reduced pressure finally afforded the desired product (44 g, 198 mmol, 93% yield). HCl was added to the free base to make handling of the compound for analytical purposes more convenient.

From corresponding hydrochloride salt, ¹H NMR (300 MHz, d3-Dimethylsulfoxide) δ (ppm): 10.28 (2H, bs), 9.07 (1H, J=1.5 Hz, bd), 8.89 (1H, J=1.5, 5.5 Hz, dd), 8.66 (1H, J=8.7 Hz, bd), 8.00-7.96 (1H, m), 7.63 (2H, J=8.5 Hz, d), 7.54 (2H, J=8.5 Hz, d), 4.38 (2H, bs), 4.24 (2H, bs).

LC/MS (Method A): Rt=0.51 min, m/z 223

### Example 5: Preparation of N-(4-ethynylbenzyl)-2,2,2-trifluoroethan-1-amine hydrochloride

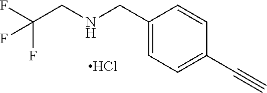

To a solution of 4-((trimethylsilyl)ethynyl)benzaldehyde (7.85 g, 37 mmol) in dry tetrahydrofuran (150 mL) was added 2,2,2-trifluoroethan-1-amine (2.92 mL, 37 mmol) and the reaction mixture stirred for 15 h at ambient temperature. Subsequently, sodium triacetoxyborohydride (39.5 g, 186 mmol) was added in portions in order to maintain the temperature of the reaction mixture below 30° C. The resulting suspension was stirred for 2.5 h after which complete conversion of the starting material was observed. Under vigorous stirring, water (50 mL) was slowly added in order to control the gas evolution. After the gas evolution had ceased, additional water (100 mL), potassium carbonate (41.2 g, 298 mmol) and methanol (200 mL) were added under vigourous stirring. When complete conversion to the desired product was ensured, the volatiles were evaporated under reduced pressure and the reaction mixture was extracted with ethyl acetate (3×150 mL). The combined organic layers were washed with aqueous saturated sodium chloride (100 mL) and concentrated to approximately 60 mL. A 4N solution of hydrochloric acid in dioxane (11 mL, 44.0 mmol) was added dropwise and the reaction mixture was stirred for 60 min. The mixture was filtered and the wet cake was washed with ethyl acetate (50 mL), with pentane (50 mL) and was dried under reduced pressure at room temperature to afford the desired product (5.99 g, 23 mmol, 62% yield).

¹H NMR (300 MHz, d3-methanol) δ (ppm): 7.55-7.48 (4H, m), 4.34 (2H, s), 4.02 (2H, J=9.1 Hz, q), 3.60 (2H, s).

LC/MS (Method A): Rt=0.98 min, m/z 214

### Example 6: Preparation of N-(4-ethynylbenzyl)-2,2,2-trifluoroethan-1-amine hydrochloride

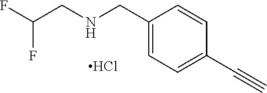

The preparation of N-(4-ethynylbenzyl)-2,2,2-trifluoroethan-1-amine hydrochloride was achieved by following steps 1 to 3 of the general method described in example 1.

¹H NMR (300 MHz, d3-methanol) δ (ppm): 7.62-7.52 (4H, m), 6.33 (1H, J=3.1, 53.8 Hz, tt), 4.35 (2H, s), 3.67 (1H, s), 3.60 (2H, J=3.1, 15.4 Hz, td).

LC/MS (Method A): Rt=0.56 min, m/z 196

### Preparation of Compounds of Formula (IV)

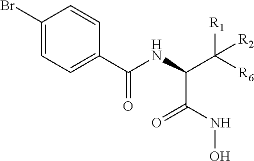

### Example 7: Preparation of tert-butyl (S)-(3-(4-bromobenzamido)-4-(hydroxyamino)-2-methyl-4-oxobutan-2-yl)carbamate

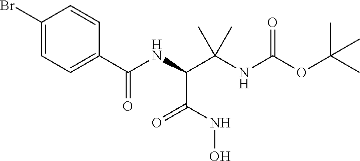 [SMILES: CC(C)(C)OC(=O)NC(C)(C)[C@H](NC(=O)c1ccc(Br)cc1)C(=O)NO]

### Step 1: Preparation of methyl (S)-2-(4-bromobenzamido)-3-((tert-butoxycarbonyl)amino)-3-methylbutanoate

A jacketed glass reactor (2 L) was charged with tetrahydrofuran (500 mL) and with 4-bromobenzoic acid (74.2 g, 365 mmol). To the suspension, oxalyl chloride (34.3 mL, 384 mmol) and dimethylformamide (0.1 mL) were sequentially added at ambient temperature under vigorous stirring. A clear solution was obtained after 18 h reaction time and full conversion was ensured by the reaction of a sample of the reaction mixture with butyl amine. The reaction mixture was added within 15 min to a cooled (0° C.) solution of methyl (S)-2-amino-3-((tert-butoxycarbonyl)amino)-3-methylbutanoate (90 g, 365 mmol) and triethylamine (102 mL, 731 mmol) in tetrahydrofuran (360 mL). After 30 min reaction time, full conversion was achieved. The reaction mixture was diluted with aqueous saturated sodium bicarbonate (1.5 L) and with methyl tert-butyl ether (1.5 L) and was stirred for 15 min. The phases were allowed to settle, and the organic layer was collected. The aqueous layer was extracted with methyl tert-butyl ether (500 mL); the combined organic layers were concentrated under reduced pressure to afford the desired product as an off-white solid (161 g, 357 mmol, 98% yield).

¹H NMR (300 MHz, d3-Dimethylsulfoxide) δ (ppm): 10.92 (1H, bs),9.02 (1H, s), 8.72 (1H, J=8.3 Hz, bd), 7.83 (2H, J=7.8 Hz, d), 7.70 (2H, J=7.8 Hz, d), 6.70 (1H, bs), 4.58 (1H, J=8.3 Hz, d), 1.36 (9H, s), 1.33 (6H, s).

LC/MS (Method A): Rt=0.51 min, m/z 223

### Step 2: Preparation of tert-butyl (S)-(3-(4-bromobenzamido)-4-(hydroxyamino)-2-methyl-4-oxobutan-2-yl)carbamate

A jacketed glass reactor (5 L) was charged with methanol (800 mL) and with methyl (S)-2-(4-bromobenzamido)-3-((tert-butoxycarbonyl)amino)-3-methylbutanoate (168 g, 356 mmol) and the resulting solution was cooled to 5° C. A 50% aqueous solution of hydroxylamine (262 mL, 4.27 mol) was added within 5 min under stirring while maintaining the temperature of the mixture below 8° C. Lithium hydroxide mono-hydrate (60 g, 1.42 mol) was added in one portion and an increase of the temperature to 17° C. was observed. The reaction mixture was stirred at ambient temperature for 30 min after which complete conversion was observed (approx. 800 mL left). The reaction mixture was diluted with water (300 mL) and the reaction mixture was concentrated under reduced pressure at 40° C. until an onset of precipitation was observed. The pH of the reaction mixture was adjusted to pH 9.0 by the addition of aqueous 1N hydrochloric acid (approx. 1.3 L) and methyl tert-butylether (2 L) was added. The resulting mixture was stirred for 15 min and the phases were allowed to settle. The organic layer was collected, and the aqueous layer was extracted with methyl tert-butylether (1 L). The combined organic layers are washed with aqueous saturated sodium chloride (500 mL) and were concentrated under reduced pressure. The obtained solid was suspended in methyl tert-butylether (800 mL), the suspension was heated to reflux for 15 min and was allowed to cool down to room temperature. The suspension was filtered, the wet cake was rinsed with diisopropylether (650 mL) and was dried under reduced pressure at 40° C. to afford the desired product as a white solid (117 g, 272 mmol, 76% yield).

¹H NMR (300 MHz, d3-Dimethylsulfoxide) δ (ppm): 10.92 (1H, bs),9.02 (1H, s), 8.72 (1H, J=8.3 Hz, bd), 7.83 (2H, J=7.8 Hz, d), 7.70 (2H, J=7.8 Hz, d), 6.70 (1H, bs), 4.58 (1H, J=8.3 Hz, d), 1.36 (9H, s), 1.33 (6H, s).

LC/MS (Method A): Rt=1.00 min, m/z 341/339

### Example 8: Preparation of 4-bromo-N-((2S,3R)-3-(tert-butoxy)-1-(hydroxyamino)-1-oxobutan-2-yl)benzamide

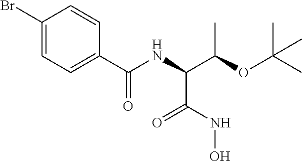 [SMILES: C[C@@H](OC(C)(C)C)[C@H](NC(=O)c1ccc(Br)cc1)C(=O)NO]

### Step 1: Preparation of methyl N-(4-bromobenzoyl)-O-(tert-butyl)-L-threoninate

4-Bromobenzoic acid (10 g, 49.7 mmol) was suspended in dry tetrahydrofuran (50 mL) at room temperature and oxalyl chloride (4.57 mL, 52.2 mmol) and a few drops of DMF are sequentially added. The resulting reaction mixture turns to a clear solution which was stirred at ambient temperature for 8 h. The reaction mixture was added within 15 min under stirring over a cooled (0° C.) suspension of methyl O-(tert-butyl)-L-threoninate hydrochloride (11.23 g, 49.7 mmol) and triethylamine (20.8 mL, 149 mmol) in dry tetrahydrofuran (150 mL) and TEA (20.80 ml, 149 mmol). After 20 min reaction time, the reaction mixture was concentrated under reduced pressure to approximately 100 mL. Aqueous saturated sodium bicarbonate (150 mL) and methyl tert-butylether (50 mL) were added and the mixture was stirred for 15 min. The phases were allowed to settle, and the organic layer was collected. The aqueous layer was extracted with methyl tert-butyl ether (50 mL) and the combined organic layers were washed with aqueous saturated sodium chloride and were concentrated under reduced pressure to afford the crude desired product which was engaged in the step 2 without further purification.

### Step 2: Preparation of 4-bromo-N-((2S,3R)-3-(tert-butoxy)-1-(hydroxyamino)-1-oxobutan-2-yl)benzamide

A jacketed glass reactor (1 L) was charged with methanol (200 mL) and with crude methyl N-(4-bromobenzoyl)-O-(tert-butyl)-L-threoninate (47.2 mmol) obtained in step 1. The resulting solution was cooled to 5° C. and 50% aqueous hydroxylamine (35 mL, 566 mmol) was slowly added, followed by lithium hydroxide mono-hydrate (7.9 g, 189 mmol). The reaction mixture was vigorously stirred at ambient temperature until a white precipitate forms (approx. 30 min reaction time). The reaction mixture was diluted with water (250 mL) and was concentrated under reduced pressure (approx. 100 mL). The pH of the mixture was adjusted to pH 9.0 by the addition of aqueous 1M hydrochloric acid. The resulting suspension was extracted methyl tert-butyl ether (500 mL and 2×100 mL). The combined organic layers were washed with aqueous saturated sodium chloride (100 ml) and concentrated under reduced pressure to afford the desired product as an off-white solid (10.9 g, 29 mmol, 62% yield).

¹H NMR (300 MHz, d3-Dimethylsulfoxide) δ (ppm): 10.66 (1H, bs), 8.94 (1H, s), 8.13 (1H, J=9.0 Hz, bd), 7.84 (2H, J=8.4 Hz, d), 7.69 (2H, J=8.4 Hz, d), 4.35 (1H, J=5.5, 9.0 Hz, dd), 3.96 (1H, J=5.5, 6.3 Hz, qd), 1.12 (9H, s), 1.08 (6H, J=6.3 Hz, d).

LC/MS (Method A): Rt=0.94 min, m/z 284/282

### Preparation of Compounds of Formula (X)

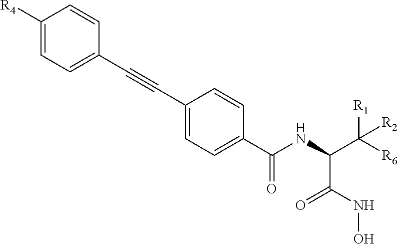

### Example 9: Preparation of tert-butyl (S)-(3-(4-((4-((cyclopropylamino)methyl)phenyl)ethynyl)benzamido)-4-(hydroxyamino)-2-methyl-4-oxobutan-2-yl)carbamate

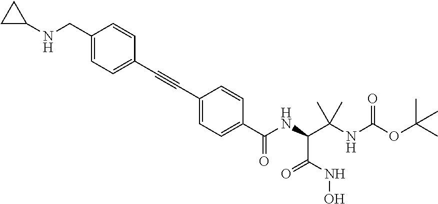 [SMILES: CC(C)(C)OC(=O)NC(C)(C)[C@H](NC(=O)c1ccc(C#Cc2ccc(CNC3CC3)cc2)cc1)C(=O)NO]

A jacketed glass reactor (2 L) was charged with anhydrous dimethylsulfoxide (1.2 L). The solvent was degassed by three vacuum/nitrogen venting cycles and tert-butyl (S)-(3-(4-bromobenzamido)-4-(hydroxyamino)-2-methyl-4-oxobutan-2-yl)carbamate (117 g, 272 mmol), N-(4-ethynylbenzyl)cyclopropanamine hydrochloride (50.8 g, 245 mmol), tetrakis(triphenylphosphine)palladium(0) (15.7 g, 13.6 mmol) and 1,8-diazabicyclo[5.4.0]undec-7-ene (143 mL, 952 mmol) were added. The temperature was raised to 70° C. and the reaction mixture was stirred at this temperature for 5 h. Heating was stopped, and the temperature was allowed to reach room temperature. The reaction mixture was transferred to a larger reactor (20 L) and ethyl acetate (6 L) and aqueous 0.5N hydrochloric acid (6 L) were added. The mixture was stirred for 15 min and the phases were allowed to settle. The aqueous layer was collected, and the organic layer was extracted aqueous 0.5N hydrochloric acid (1.2 L). The pH of the combined aqueous layers was set to pH 6.0 by the addition of aqueous saturated sodium carbonate (approx. 0.5 L), and the combined aqueous layers were extracted with ethyl acetate (6 L and 1.2 L). The combined organic layers were back extracted aqueous saturated sodium bicarbonate (1.2 L), washed with aqueous saturated sodium chloride (1.2 L) and were concentrated under reduced pressure to afford the desired product as a light yellow solid (118.4 g, 225 mmol, 92% yield).

¹H NMR (300 MHz, d3-Dimethylsulfoxide) δ (ppm): 7.92 (2H, J=8.3 Hz, bd), 7.64 (2H, J=8.3 Hz, bd), ), 7.54 (2H, J=8.3 Hz, bd), ), 7.41 (2H, J=8.3 Hz, bd), 4.72 (1H, s), 3.85 (2H, s), 2.18-2.14 (1H, m), 1.50 (3H, s), 1.47 (9H, s), 1.43 (3H, s), 0.52-0.49 (2H, m), 0.43-0.41 (2H, m).

LC/MS (Method A): Rt=0.79 min, m/z 521

### Example 10: Preparation of tert-butyl (S)-(4-(hydroxyamino)-3-(4-((4-(((2-methoxyethyl)amino)methyl)phenyl)ethynyl)benzamido)-2-methyl-4-oxobutan-2-yl)carbamate

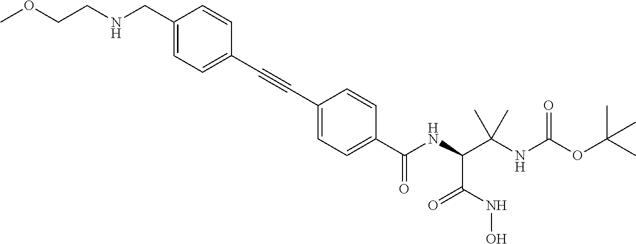 [SMILES: COCCNCc1ccc(C#Cc2ccc(C(=O)N[C@H](C(=O)NO)C(C)(C)NC(=O)OC(C)(C)C)cc2)cc1]

A jacketed glass reactor (1 L) was charged with anhydrous dimethylsulfoxide (210 mL), tert-butyl (S)-(3-(4-bromobenzamido)-4-(hydroxyamino)-2-methyl-4-oxobutan-2-yl)carbamate (20.5 g, 47.6 mmol), N-(4-ethynylbenzyl)-2-methoxyethan-1-amine hydrochloride (9.68 g, 42.9 mmol), tetrakis(triphenylphosphine)palladium(0) (2.75 g, 2.4 mmol), copper(I) iodide (0.90 g, 4.8 mmol) and 1,8-diazabicyclo[5.4.0]undec-7-ene (25.1 ml, 167 mmol). The temperature was raised to 70° C. and the reaction mixture was stirred at this temperature for 1 h. Heating was stopped, and the reaction mixture was allowed to cool to ambient temperature. The mixture was transferred to a larger reactor (3 L) and ethyl acetate (800 mL) and aqueous 0.5N hydrochlorid acid (800 mL) were added. The mixture was stirred for 15 min and the phases were allowed to settle. The aqueous layer was collected, and the organic layer was extracted with aqueous 0.5N hydrochlorid acid (400 mL). The combined aqueous layers were washed with ethyl acetate (200 mL) and the pH was adjusted to pH 6 by the addition of aqueous saturated sodium carbonate. The aqueous layer was extracted with ethyl acetate (2×600 mL), was washed with aqueous saturated sodium chloride (200 mL) and concentrated under reduced pressure to afford a grey solid. Purification by column chromatography on silica gel (eluent dichloromethane:methanol 90:10) affords the desired product as a beige solid (18.8 g, 34.8 mmol, 81% yield).

LC/MS (Method A): Rt=0.74 min, m/z 539

### Example 11: Preparation of tert-butyl (S)-(4-(hydroxyamino)-2-methyl-4-oxo-3-(4-((4-(((pyridin-4-ylmethyl)amino)methyl)phenyl)ethynyl)benzamido)butan-2-yl)carbamate

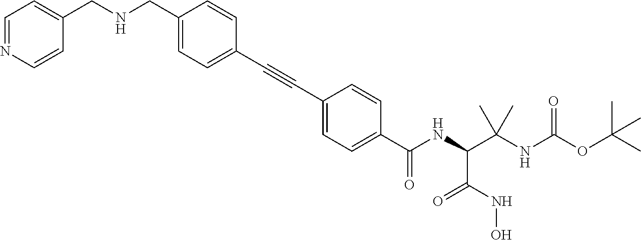 [SMILES: CC(C)(C)OC(=O)NC(C)(C)[C@H](NC(=O)c1ccc(C#Cc2ccc(CNCc3ccncc3)cc2)cc1)C(=O)NO]

A jacketed glass reactor (500 mL) was charged with anhydrous degased dimethylsulfoxide (150 mL), tert-butyl (S)-(3-(4-bromobenzamido)-4-(hydroxyamino)-2-methyl-4-oxobutan-2-yl)carbamate (28.5 g, 66 mmol), N-(4-ethynylbenzyl)-1-(pyridin-4-yl)methanamine (15.5 g, 64 mmol), tetrakis(triphenylphosphine)palladium(0) (3.83 g, 3.3 mmol) and 1,8-diazabicyclo[5.4.0]undec-7-ene (34.9 mL, 232 mmol). The temperature was raised to 50° C. and copper (I) iodide (1.26 g, 6.6 mmol) was added under vigorous stirring in portions within 10 min. After completion of the addition, the temperature was set to 70° C. and the reaction mixture was stirred for 30 min at this temperature below it was allowed to cool to ambient temperature. The reaction mixture was poured onto water (800 mL) under stirring and the formed precipitate was filtered. Ethyl acetate (800 mL) was added to the filtrate and the resulting mixture was stirred for 15 min. The phases were allowed to settle, and the organic layer was collected. The aqueous layer was extracted with ethyl acetate (2×100 mL), the combined organic layers were washed with aqueous saturated sodium chloride (100 mL) and were concentrated under reduced pressure. Purification by column chromatography on silica gel (eluent ethyl acetate) affords the desired product as a beige solid (22.7 g, 36.0 mmol, 56% yield).

¹H NMR (300 MHz, d3-Dimethylsulfoxide) δ (ppm): 10.92 (1H, bs), 9.03 (1H, s), 8.73 (1H, J=9.0 Hz, bd), 8.50 (2H, J=5.9 Hz, bd), 7.93 (2H, J=8.2 Hz, bd), 7.55 (2H, J=8.2 Hz, bd), 7.43 (2H, J=8.2 Hz, bd), 7.38 (2H, J=5.9 Hz, bd), 6.73 (1H, s), 4.60 (1H, J=8.9 Hz, bd), 3.72 (4H, m), 3.34 (1H, m), 2.55 (3H, s), 1.37 (9H, bs), 1.34 (3H, bs), 1.33 (3H, bs).

LC/MS (Method A): Rt=0.70 min, m/z 572

### Example 12: Preparation of tert-butyl (S)-(4-(hydroxyamino)-2-methyl-4-oxo-3-(4-((4-(((pyridin-3-ylmethyl)amino)methyl)phenyl)ethynyl)benzamido)butan-2-yl)carbamate

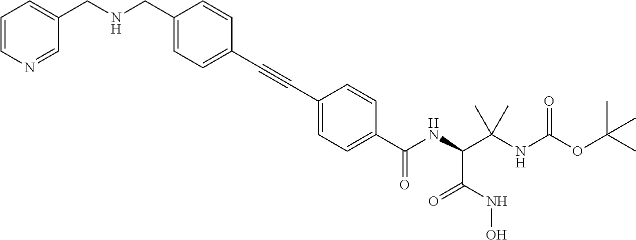 [SMILES: CC(C)(C)OC(=O)NC(C)(C)[C@H](NC(=O)c1ccc(C#Cc2ccc(CNCc3cccnc3)cc2)cc1)C(=O)NO]

A jacketed glass reactor (500 mL) was charged with anhydrous degased dimethylsulfoxide (150 mL), tert-butyl (S)-(3-(4-bromobenzamido)-4-(hydroxyamino)-2-methyl-4-oxobutan-2-yl)carbamate (29.5 g, 68.6 mmol), N-(4-ethynylbenzyl)-1-(pyridin-3-yl)methanamine (14.8 g, 66.5 mmol), tetrakis(triphenylphosphine)palladium(0) (3.96 g, 3.43 mmol) and 1,8-diazabicyclo[5.4.0]undec-7-ene (36.2 mL, 240 mmol). The temperature of the resulting mixture was set to 50° C. and copper(I) iodide (1.31 g, 6.86 mmol) was added in portions under stirring. After 40 min reaction time, full conversion was observed, and the reaction mixture was allowed to reach room temperature. The reaction mixture was poured onto water (500 mL) under stirring and the pH was set to neutral by the addition of aqueous 3N hydrochloric acid (approx. 55 mL). The formed precipitate was filtered, ethyl acetate (500 mL) was added to the filtrate and the mixture was stirred for 15 min. The phases were allowed to settle, and the organic layer was collected. The aqueous layer was extracted with ethyl acetate (2×100 mL), the combined organic layers were washed with aqueous saturated sodium chloride (100 mL) and concentrated under reduced pressure to afford the desired product (36 g, 59.8 mmol, 90% yield).

¹H NMR (300 MHz, d3-methanol) δ (ppm): 8.55 (1H, bs), 8.48 (1H, bs), 7.93-7.87 (3H, m), 7.64 (2H, J=8.1 Hz, bd), 7.55 (2H, J=8.1 Hz, bd), 7.46-7.40 (3H, m), 4.72 (1H, s), 3.83 (4H, bs), 1.50 (3H, s), 1.47 (9H, s), 1.44 (3H, s).

LC/MS (Method A): Rt=0.72 min, m/z 572

### Example 13: Preparation of tert-butyl (S)-(3-(4-((4-(((2,2-difluoroethyl)amino)methyl)phenyl)ethynyl)benzamido)-4-(hydroxyamino)-2-methyl-4-oxobutan-2-yl)carbamate

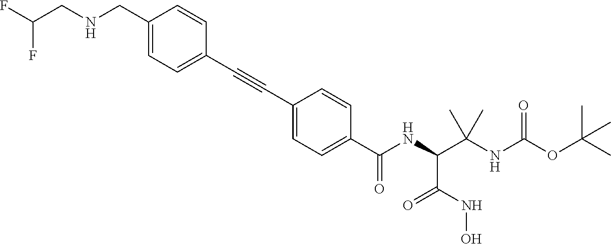 [SMILES: CC(C)(C)OC(=O)NC(C)(C)[C@H](NC(=O)c1ccc(C#Cc2ccc(CNCC(F)F)cc2)cc1)C(=O)NO]

The preparation of tert-butyl (S)-(3-(4-((4-(((2,2-difluoroethyl)amino)methyl)phenyl)ethynyl)benzamido)-4-(hydroxyamino)-2-methyl-4-oxobutan-2-yl)carbamate was achieved by following the general method described in example 9. ¹H NMR (300 MHz, d3-methanol) δ (ppm): 8.55 (1H, bs), 8.48 (1H, bs), 7.93-7.87 (3H, m), 7.64 (2H, J=8.1 Hz, bd), 7.55 (2H, J=8.1 Hz, bd), 7.46-7.40 (3H, m), 4.72 (1H, s), 3.83 (4H, bs), 1.50 (3H, s), 1.47 (9H, s), 1.44 (3H, s).

LC/MS (Method A): Rt=0.75 min, m/z 545

### Example 14: Preparation of N-((2S,3R)-3-(tert-butoxy)-1-(hydroxyamino)-1-oxobutan-2-yl)-4-((4-(((2,2,2-trifluoroethyl)amino)methyl)phenyl)ethynyl)benzamide

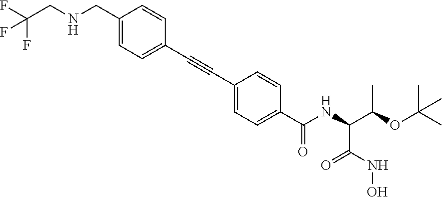 [SMILES: C[C@@H](OC(C)(C)C)[C@H](NC(=O)c1ccc(C#Cc2ccc(CNCC(F)(F)F)cc2)cc1)C(=O)NO]

The preparation of N-((2S,3R)-3-(tert-butoxy)-1-(hydroxyamino)-1-oxobutan-2-yl)-4-((4-(((2,2,2-trifluoroethyl)amino)methyl)phenyl)ethynyl)benzamide was achieved by following the general method described in example 9.

LC/MS (Method A): Rt=1.07 min, m/z 506

### Preparation of Compounds of Formula (I)

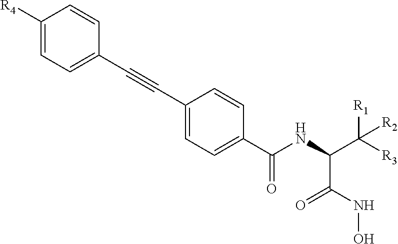

### Example 15: Preparation of (S)-N-(3-amino-1-(hydroxyamino)-3-methyl-1-oxobutan-2-yl)-4-((4-((cyclopropylamino)methyl)phenyl)ethynyl)benzamide dihydrochloride

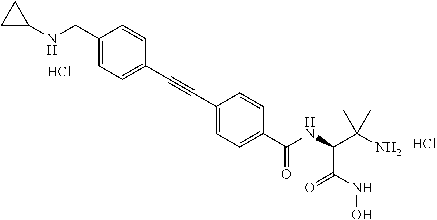 [SMILES: CC(C)(N)[C@H](NC(=O)c1ccc(C#Cc2ccc(CNC3CC3)cc2)cc1)C(=O)NO ; Cl ; Cl]

A jacketed glass reactor (1 L) was charged with acetonitrile (300 mL), water (50 mL) and with tert-butyl (S)-(3-(4-((4-((cyclopropylamino)methyl)phenyl)ethynyl)benzamido)-4-(hydroxyamino)-2-methyl-4-oxobutan-2-yl)carbamate (90 g, 171 mmol). Aqueous 12N hydrochloric acid (141 mL, 1.71 mol) was added at 25° C. within 5 min and an increase fo the temperature to 33° C. was observed. After 3 h reaction time, full conversion was achieved and the reaction mixture was added within 20 min to acetone (4.5 L) under vigorous stirring at about 50° C. A suspension was obtained and was heated to reflux for 10 min after which the temperature was cooled to 25° C. and stirring continued for 15 h. The precipitate was filtered off, was washed with acetone (100 mL) and was dried under reduced pressure at 40° C. to afford the desired product as a light yellow solid (73.7 g, 147 mmol, 86% yield).

¹H NMR (300 MHz, d3-Dimethylsulfoxide) δ (ppm): 11.29 (1H, s), 9.89 (1H, bs), 9.24 (1H, bs), 8.86 (1H, J=9.3 Hz, bd), 8.38 (3H, bs), 8.10 (2H, J=8.4 Hz, bd), 7.71-7.62 (6H, m), 4.70 (1H, J=9.3 Hz, bd), 4.28-4.19 (2H, m), 2.62 (1H, bs), 1.38 (3H, s), 1.36 (3H, s), 0.99-0.94 (2H, m), 0.74-0.67 (2H, m).

LC/MS (Method A): Rt=0.53 min, m/z 421

### Example 16: Preparation of N-((2S,3R)-3-hydroxy-1-(hydroxyamino)-1-oxobutan-2-yl)-4-((4-(((2,2,2-trifluoroethyl)amino)methyl)phenyl)ethynyl)benzamide

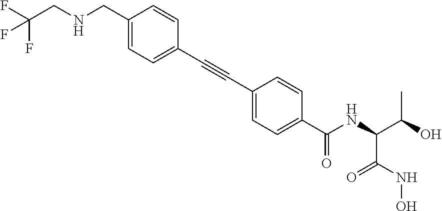 [SMILES: C[C@@H](O)[C@H](NC(=O)c1ccc(C#Cc2ccc(CNCC(F)(F)F)cc2)cc1)C(=O)NO]

Aqueous 12N hydrochloric acid (10 mL, 120 mmol) was added dropwise to a stirred suspension of N-((2S,3R)-3-(tert-butoxy)-1-(hydroxyamino)-1-oxobutan-2-yl)-4-((4-(((2,2,2-trifluoroethyl)amino)methyl)phenyl)ethynyl)benzamide (3.09 g, 6.11 mmol) in acetonitrile (30 mL). The suspended solids dissolve and the resulting solution was stirred for 30 min at ambient temperature after which complete conversion was observed. The reaction mixture was diluted with water (100 mL), the pH was adjusted between 8 to 9 by the addition of aqueous saturated sodium bicarbonate and ethyl acetate (250 mL) was added. The resulting mixture was stirred for 15 min and the phases were allowed to settle. The organic layer was collected, and the aqueous layer was extracted with ethyl acetate (250 mL). The combined organic layers were washed with aqueous saturated sodium hydrogencarbonate (100 mL), with aqueous sodium chloride (100 mL) brine and were concentrated under reduced pressure. The obtained crude product was purified by recrystallization from ethyl acetate (approx. 100 mL), followed by washing of the wet cake with methyl tert-butyl ether (20 mL) and drying under reduced pressure at 40° C. The desired product as an off-white solid (2.14 g, 4.76 mmol, 78% yield).

¹H NMR (300 MHz, d3-Dimethylsulfoxide) δ (ppm): 10.70 (1H, bs), 8.87 (1H, bs), 8.16 (1H, J=8.7 Hz, d), 7.96 (2H, J=8.3 Hz, d), 7.66 (2H, J=8.3 Hz, d), 7.56 (2H, J=8.3 Hz, d), 7.42 (2H, J=8.3 Hz, d), 4.28 (1H, J=5.5, 8.3 Hz, dd), 4.04 (1H, J=5.5, 6.9 Hz, qd), 3.83 (2H, J=6.9 Hz, bd), 3.24-3.18 (2H, m), 3.04-2.99 (2H, m), 1.10 (3H, J=6.3 Hz, d).

LC/MS (Method A): Rt=0.80 min, m/z 450

### Example 17: Preparation of (S)-N-(3-amino-1-(hydroxyamino)-3-methyl-1-oxobutan-2-yl)-4-((4-(((2-methoxyethyl)amino)methyl)phenyl)ethynyl)benzamide dihydrochloride

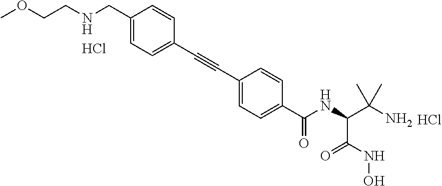 [SMILES: COCCNCc1ccc(C#Cc2ccc(C(=O)N[C@H](C(=O)NO)C(C)(C)N)cc2)cc1 ; Cl ; Cl]

Aqueous 12N hydrochloric acid (55 mL, 666 mmol) was added within 5 min at ambient temperature to a stirred solution of tert-butyl (S)-(4-(hydroxyamino)-3-(4-((4-(((2-methoxyethyl)amino)methyl)phenyl)ethynyl)benzamido)-2-methyl-4-oxobutan-2-yl)carbamate (20 g, 33 mmol) in a mixture of acetonitrile (180 mL) and water (20 mL). After 3 h reaction time, complete conversion was observed. The volatiles were evacuated by distillation under reduced pressure and the obtained residue was diluted in dioxane (200 mL) was freeze dried to deliver the desired product as a pale yellow solid (20.8 g, 40 mmol, quant.).

¹H NMR (300 MHz, d3-Dimethylsulfoxide) δ (ppm): 11.27 (1H, s), 9.51 (2H, bs), 8.82 (1H, J=9.0 Hz, bd), 8.30 (3H, bs), 8.09 (2H, J=8.4 Hz, d), 7.70-7.66 (6H, m), 4.70 (1H, J=9.0 Hz, d), 4.21-4.18 (2H, m), 3.65 (2H, J=5.1 Hz, bt), 3.30 (3H, s), 3.11-3.03 (2H, m), 1.37 (3H, s), 1.35 (3H, s).

LC/MS (Method A): Rt=0.51 min, m/z 439

### Example 18: Preparation of (S)-N-(3-amino-1-(hydroxyamino)-3-methyl-1-oxobutan-2-yl)-4-((4-(((2,2-difluoroethyl)amino)methyl)phenyl)ethynyl)benzamide

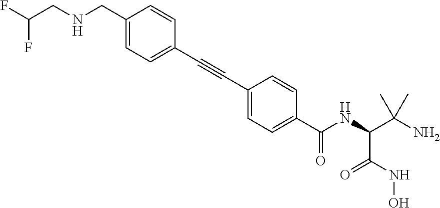 [SMILES: CC(C)(N)[C@H](NC(=O)c1ccc(C#Cc2ccc(CNCC(F)F)cc2)cc1)C(=O)NO]

The preparation of(S)-N-(3-amino-1-(hydroxyamino)-3-methyl-1-oxobutan-2-yl)-4-((4-(((2,2-difluoroethyl)amino)methyl)phenyl)ethynyl)benzamide was achieved by following the general method described in example 15.

¹H NMR (300 MHz, d3-methanol) δ (ppm): 7.90 (2H, J=6.3 Hz, d), 7.63 (2H, J=6.3 Hz, d), 7.54 (2H, J=6.2 Hz, d), 7.40 (2H, J=6.2 Hz, d), 5.93 (1H, J=3.2, 40.8 Hz, tt), 4.48 (1H, s), 3.86 (2H, s), 2.92 (2H, J=3.2, 11.6 Hz, td), 1.31 (3H, s), 1.22 (3H, s).

LC/MS (Method C): Rt=7.66 min, m/z 445

### Example 19: Preparation of (S)-N-(3-amino-1-(hydroxyamino)-3-methyl-1-oxobutan-2-yl)-4-((4-(((2,2,2-trifluoroethyl)amino)methyl)phenyl)ethynyl)benzamide

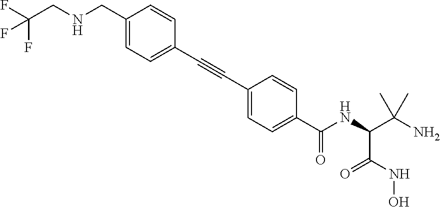 [SMILES: CC(C)(N)[C@H](NC(=O)c1ccc(C#Cc2ccc(CNCC(F)(F)F)cc2)cc1)C(=O)NO]

The preparation of(S)-N-(3-amino-1-(hydroxyamino)-3-methyl-1-oxobutan-2-yl)-4-((4-(((2,2,2-trifluoroethyl)amino)methyl)phenyl)ethynyl)benzamide was achieved by following the general method described in example 15.

¹H NMR (300 MHz, d3-methanol) δ (ppm): 7.88 (2H, J=8.1 Hz, d), 7.59 (2H, J=8.1 Hz, d), 7.51 (2H, J=8.1 Hz, d), 7.38 (2H, J=8.1 Hz, d), 4.50 (1H, s), 3.87 (2H, s), 3.18 (2H, J=9.9 Hz, q), 1.31 (3H, s), 1.23 (3H, s).

LC/MS (Method C): Rt=8.47 min, m/z 463.

1. A method of preparing a compound of Formula (I) 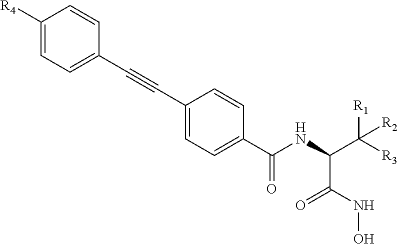
or salt thereof, comprising
converting a compound of Formula (III) 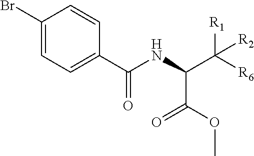
to a compound of Formula (IV) 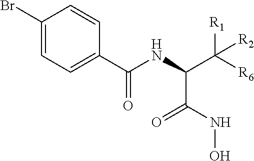
and coupling the compound of Formula (IV) with a compound of Formula (IX) 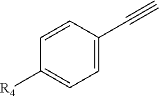
or a salt thereof to yield the compound of Formula (I) with less than 2% of a compound of Formula (XI) 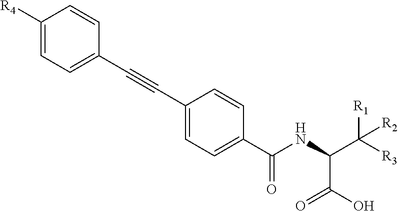
produced as a byproduct, wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CF₂, —CF₃, 3-pyridinyl or 4-pyridinyl; and
R⁶ is —NHC(O)OC(CH₃)₃ or —OC(CH₃)₃.

2. The method of claim 1, comprising the step of reacting the compound of Formula (III) 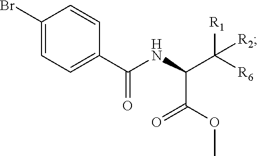
with hydroxylamine in the presence of lithium hydroxide to produce the compound of Formula (IV)

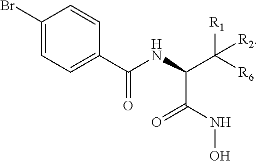

3. The method of claim 1 comprising the step of coupling the compound of Formula (IV) 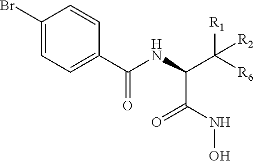
to the compound of Formula (IX) 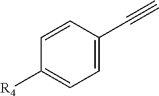
or a salt thereof
in the presence of a palladium catalyst, a base and optionally copper iodide to yield a compound of Formula (X) 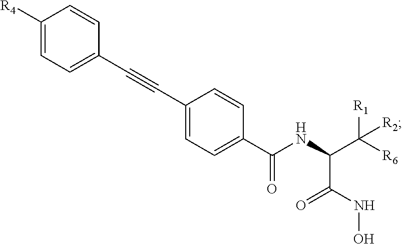
and then subsequently converting the compound of Formula (X) to
the compound of Formula (I) or a salt thereof.

4. The method of claim 1, comprising the step of forming the compound of Formula (III)
by reacting a compound of Formula (II) 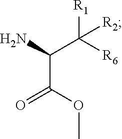
with 4 bromo benzoic acid to yield a compound of Formula (III).

5. The method of claim 1 comprising the steps of
a) reacting a compound of Formula (II) 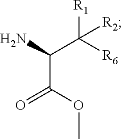
with 4 bromo benzoic acid to yield a compound of Formula (III) 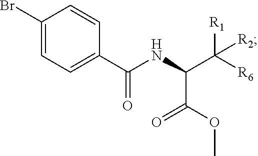
b) reacting the compound of Formula (III) with hydroxylamine in the presence of lithium hydroxide to produce a compound of Formula (IV) 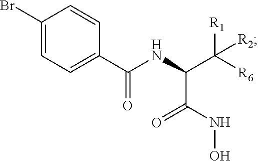
c) reacting the compound of Formula (IV) with a compound of Formula (IX) 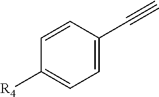
or a salt thereof
in the presence of a palladium catalyst, a base and optionally copper iodide to yield a compound of Formula (X) 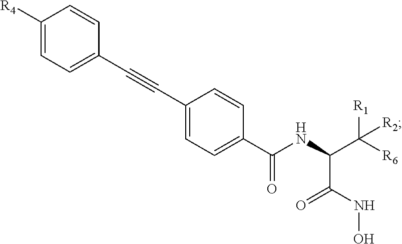
and
d) reacting the compound of Formula (X) with an acid to yield the compound of Formula (I) or a salt thereof
wherein
R¹ and R² are each independently hydrogen or C₁ to C₄ alkyl;
R³ is NH₂, or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

6. The method of claim 3, wherein the palladium catalyst is Pd(PPh₃)₄.

7. The method of claim 1, wherein the compound of Formula (I) is selected from the group consisting of

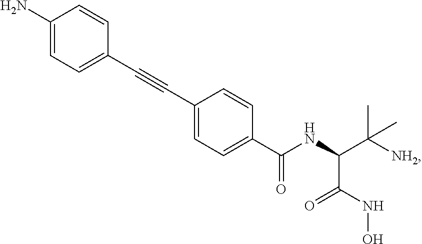 [SMILES: CC(C)(N)[C@H](NC(=O)c1ccc(C#Cc2ccc(N)cc2)cc1)C(=O)NO]

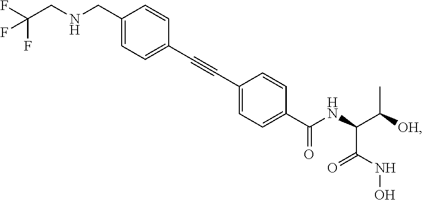 [SMILES: C[C@@H](O)[C@H](NC(=O)c1ccc(C#Cc2ccc(CNCC(F)(F)F)cc2)cc1)C(=O)NO]

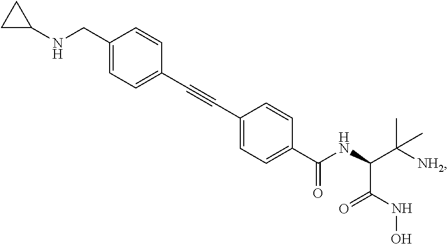 [SMILES: CC(C)(N)[C@H](NC(=O)c1ccc(C#Cc2ccc(CNC3CC3)cc2)cc1)C(=O)NO]

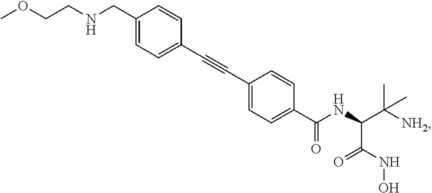 [SMILES: COCCNCc1ccc(C#Cc2ccc(C(=O)N[C@H](C(=O)NO)C(C)(C)N)cc2)cc1]

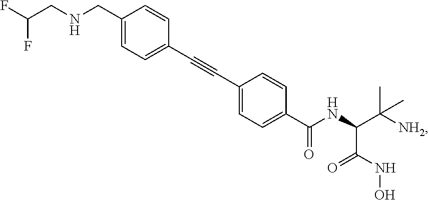 [SMILES: CC(C)(N)[C@H](NC(=O)c1ccc(C#Cc2ccc(CNCC(F)F)cc2)cc1)C(=O)NO]

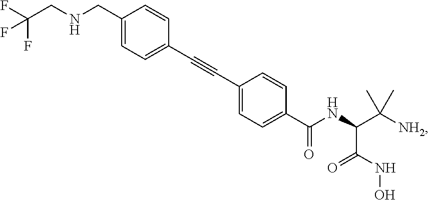 [SMILES: CC(C)(N)[C@H](NC(=O)c1ccc(C#Cc2ccc(CNCC(F)(F)F)cc2)cc1)C(=O)NO]

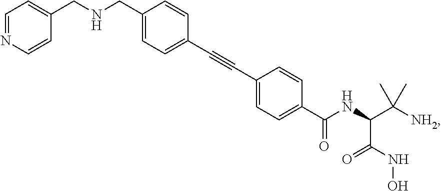 [SMILES: CC(C)(N)[C@H](NC(=O)c1ccc(C#Cc2ccc(CNCc3ccncc3)cc2)cc1)C(=O)NO]

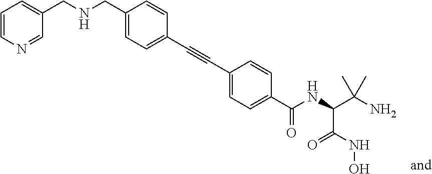 [SMILES: CC(C)(N)[C@H](NC(=O)c1ccc(C#Cc2ccc(CNCc3cccnc3)cc2)cc1)C(=O)NO]

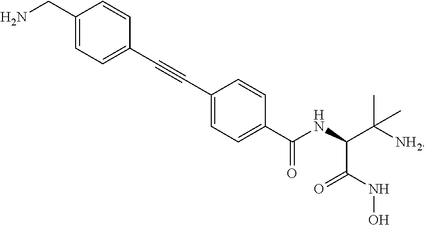 [SMILES: CC(C)(N)[C@H](NC(=O)c1ccc(C#Cc2ccc(CN)cc2)cc1)C(=O)NO]

8. The method of claim 1, wherein the compound of Formula (I) is a hydrobromide salt, a dihydrobromide salt, a hydrochloride salt or a dihydrochloride salt.

9. A compound of Formula (X) 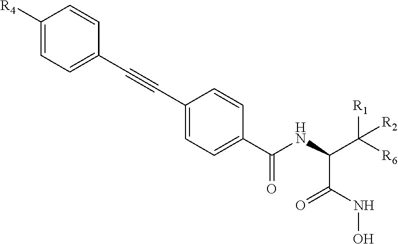
or a salt thereof
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃ or -3-pyridinyl or 4-pyridinyl; and
R⁶ is —NHC(O)OC(CH₃)₃ or —OC(CH₃)₃.

10. The compound of claim 9, wherein the compound of Formula X is selected from the group consisting of 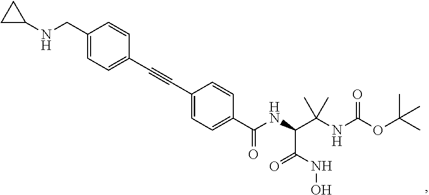 [SMILES: CC(C)(C)OC(=O)NC(C)(C)[C@H](NC(=O)c1ccc(C#Cc2ccc(CNC3CC3)cc2)cc1)C(=O)NO] 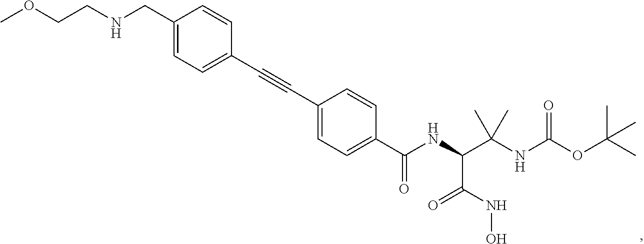 [SMILES: COCCNCc1ccc(C#Cc2ccc(C(=O)N[C@H](C(=O)NO)C(C)(C)NC(=O)OC(C)(C)C)cc2)cc1] 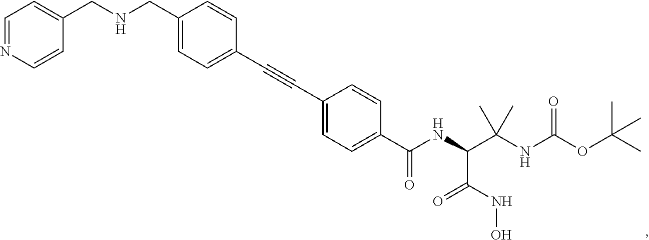 [SMILES: CC(C)(C)OC(=O)NC(C)(C)[C@H](NC(=O)c1ccc(C#Cc2ccc(CNCc3ccncc3)cc2)cc1)C(=O)NO] 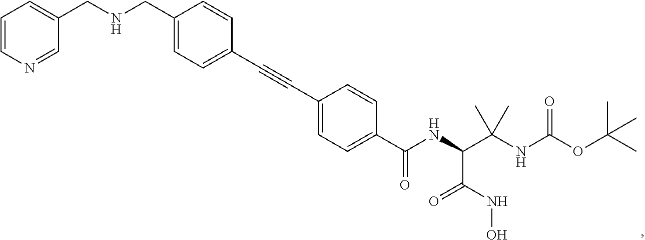 [SMILES: CC(C)(C)OC(=O)NC(C)(C)[C@H](NC(=O)c1ccc(C#Cc2ccc(CNCc3cccnc3)cc2)cc1)C(=O)NO] 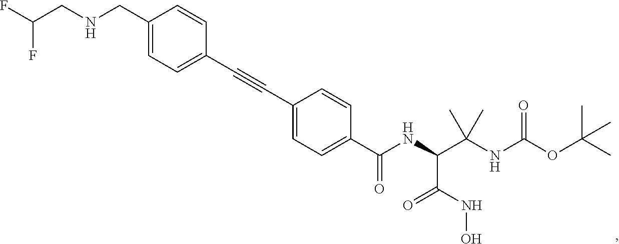 [SMILES: CC(C)(C)OC(=O)NC(C)(C)[C@H](NC(=O)c1ccc(C#Cc2ccc(CNCC(F)F)cc2)cc1)C(=O)NO] 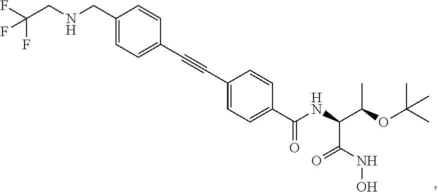 [SMILES: C[C@@H](OC(C)(C)C)[C@H](NC(=O)c1ccc(C#Cc2ccc(CNCC(F)(F)F)cc2)cc1)C(=O)NO] 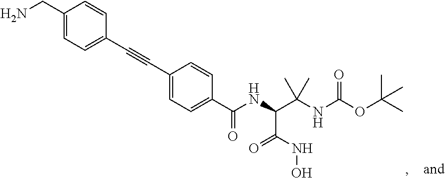 [SMILES: CC(C)(C)OC(=O)NC(C)(C)[C@H](NC(=O)c1ccc(C#Cc2ccc(CN)cc2)cc1)C(=O)NO] 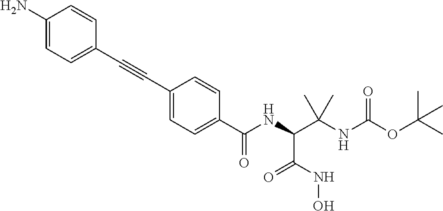 [SMILES: CC(C)(C)OC(=O)NC(C)(C)[C@H](NC(=O)c1ccc(C#Cc2ccc(N)cc2)cc1)C(=O)NO]
or salt thereof.

11. The compound of claim 9, wherein the compound is a hydrobromide salt, a dihydrobromide salt, a hydrochloride salt or a dihydrochloride salt.

12. A method of preparing a compound of claim 9 comprising reacting a compound of Formula (IX) 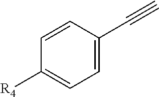
or salt thereof
with a compound of Formula (IV) 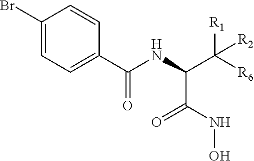
in the presence of a palladium catalyst, a base and optionally copper iodide to yield a compound of Formula (X) and optionally converting it to a salt
wherein
R¹ and R² are each independently hydrogen or C₁ to C₄ alkyl optionally substituted with NH₂ or OH;
R⁴ is NH₂ or CH₂—NH—R⁵;
R⁵ is hydrogen, cyclopropyl or —CH₂— substituted by —CH₂OCH₃, —CHF₂, —CF₃, 3-pyridinyl or 4-pyridinyl; and
R⁶ is —NHC(O)OC(CH₃)₃ or —OC(CH₃)₃.

13. The method of claim 12, wherein the palladium catalyst is Pd(PPh₃)₄.

14. A compound of Formula (IX) 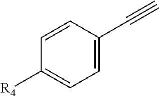
or a salt thereof, wherein
R⁴ is CH₂—NH—R⁵ or NH₂;
R⁵ is hydrogen, cyclopropyl or —CH₂— substituted by —CH₂OCH₃, —CHF₂, —CF₃, 3-pyridinyl or 4-pyridinyl.

15. The compound of claim 14, wherein the compound of Formula IX is selected from the group consisting of 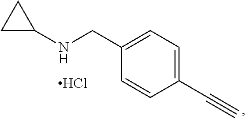 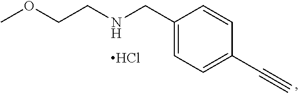 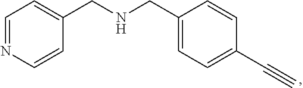 [SMILES: C#Cc1ccc(CNCc2ccncc2)cc1] 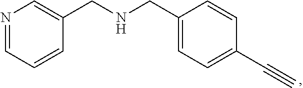 [SMILES: C#Cc1ccc(CNCc2cccnc2)cc1] 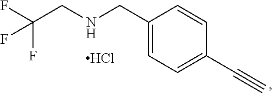 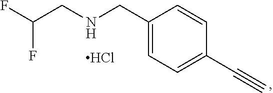 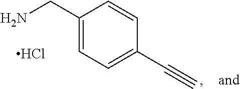 [chemical structure]
